(19) United States
(12) Patent Application Publication
(10) Pub. No.: US 2017/0300631 A1
(43) Pub. Date: Oct. 19, 2017

(54) METHOD TO ESTIMATE REAL NOISE EXPOSURE LEVELS

(71) Applicant: Bertrand Johnson Acoustique Inc., MONTREAL (CA)

(72) Inventors: Robert A. BERTRAND, LAVAL (CA); Zhaoxing HUANG, Saint-Laurent (CA); Zhifeng ZHANG, Brossard (CA); Hrair TORIKIAN, LAVAL (CA)

(73) Assignee: Bertrand Johnson Acoustique Inc., MONTREAL (CA)

(21) Appl. No.: 15/486,286

(22) Filed: Apr. 12, 2017

Related U.S. Application Data
(60) Provisional application No. 62/321,444, filed on Apr. 12, 2016.

(51) Int. Cl. G06F 19/00; A61B 5/12; A61B 5/00

(52) U.S. Cl. CPC G06F 19/322; G06F 19/3431; A61B 5/125; A61B 5/7275

(57) ABSTRACT

There is provided a method for determining a noise exposure level associated as the cause of an observed evolution of hearing acuity of an individual of known gender. The method comprises the following steps: 1) providing a first audiogram of the individual measured at age X and a second audiogram of the individual measured at age Y; 2) inputting the individual's gender, age X, and a time period equal to Y−X in a statistical hearing threshold levels evolution prediction formula; 3) calculating projected hearing loss audiograms specific to each of a plurality of possible noise level exposure values, using the prediction formula; 4) comparing a pattern of each calculated projected audiogram with a pattern the second audiogram; 5) selecting the projected audiogram that best fits the second audiogram; and 6) assuming that the noise exposure level value associated with the selected projected audiogram is the noise exposure value that caused the evolution of hearing acuity observed between the first and the second audiograms. There is also provided systems for performing the method and methods for providing services to clients or enabling users regarding determination of real ear noise exposure values.

### CROSS-REFERENCE TO RELATED APPLICATION

This application claims priority from U.S. patent provisional application 62/321,444 filed Apr. 12, 2016, the specification of which is hereby incorporated herein by reference in its entirety.

### BACKGROUND

### (a) Field

This invention relates generally to the field of industrial hygiene and more particularly in relation to the attenuation of hearing protection devices (HPDs) for subjects exposed to high noise levels inducing hearing loss. The subject matter disclosed generally relates to the attenuation provided in the field by HPDs and audiometry in relation to the evolution of hearing threshold level (HTL) frequencies.

More specifically, the subject matter disclosed relates to methods and systems for estimating the real ear exposure levels to which an individual is exposed regardless of the HPD worn and the manner or duration for which it is worn.

The disclosure is further concerned with a method of predicting future hearing loss parameters as a function of observed audiometric hearing thresholds and patterns. The method includes considerations relating to the aging factor which is present in all individuals.

### (b) Related Prior Art

Epidemiological studies have demonstrated a relationship between the degree of hearing loss taking into consideration the age, gender, levels of noise exposure and duration of exposure. It is well recognized that exposure to noise may cause hearing loss in workers. The risk of occupational noise induced hearing loss (ONIHL) must be managed as hearing loss is the second most prevalent health issue globally. Some types of work conditions are particularly prone to expose workers to noise conditions that are likely to lead to some level of hearing loss. Miners and workers in many manufacturing industries are examples of the many individuals subjected to high noise exposure levels and ONIHL.

Due to the effect of noise upon hearing, HPDs are used to decrease the noise level intensity to which a worker is exposed. HPD attenuation is evaluated by either the mandatory Noise Reduction Ratio (NRR) method enacted by OSHA in the United States of America (US) or High-Medium-Low (HML) method of the International Standard Organization (ISO). The laboratory attenuation is evaluated for each frequency or series of frequencies and a global attenuation is obtained for each HPD using the NRR method, or for groups of frequencies using the HML method.

In Hearing Conservation Programs (HCPs), education and training have always been important parts of hearing loss prevention. This includes training regarding noise exposure and adequate use of hearing protection. Unfortunately, it is difficult to convince every person or worker exposed to noise that hearing protection is in their own long range interest. Even when hearing protection is worn, it is a difficult task for hearing conservation professionals to estimate hearing protection performance. Two considerations are difficult to ascertain: the proper use of the HPD and the time during which an HPD is worn. Several factors such as improper use of the HPD or time it is worn when exposed to noise may alter the daily attenuation of the HPD.

At present, two standards are available to predict ONIHL based on the age, gender, level and duration of exposure. They are the ISO1999 and ANSI S3.44 standards. Both the ISO1999 and ANSI S3.44 include the aging factor based on the ISO7029 standard. The ISO7029 standard presents predication of presbycusis for the frequencies of 0.5, 1, 2, 3, 4, 6 and 8 kHz for both males and females from the age of 18 to 70. This standard has 2 annexes: Annex A for otologically normal subjects/individuals and Annex B consists of an unscreened population. A definition of otologically normal subject is included in the ISO7029 standard in Annex A. The standardized procedures for rating hearing protection devices, such as NRR and HML are of a statistical nature for groups of people, not for individuals. These ratings cannot integrate the daily proper use of HPD for a specific individual.

Intrinsic and Extrinsic factors must be taken into account in relation to abnormal evolution of HTLs. As far as intrinsic factors are concerned, there is always the constant presence of the presbycusis factors. Other intrinsic factors to be considered are genetic hearing loss for which the evolution of HTLs may evolve independent of the presence of exposure to noise or the use of HPDs. Otological diseases may modify the evolution of HTLs as well as certain systemic diseases especially diseases that affect vascularization of the auditory system either at the central and/or peripheral portion.

Extrinsic causes may include incidents such as trauma, use of ototoxic drugs; barotrauma and ototoxic environment are the main causes of extrinsic origin.

The presence of these intrinsic or extrinsic causes may be easily detected by the pre-screening test questionnaire performed before each audiometric data accumulation. On the other hand, certain of these intrinsic or extrinsic factors may be suspected in relation to sudden abnormal evolution of HTLs that produce unacceptable frequency levels that may be an indication to proceed to identify the cause of such abnormal variation.

There is a long tradition of data gathering about hearing conditions of workers. Workers generally have an audiometric screening test performed prior to hiring and at some intervals throughout their work history. A huge amount of data is thus available to analyze and interpret audiometric tests reports. Based on such data, and on information about known noise exposure levels and duration of exposure, individual gender and age, previous research work led to the prediction of HTLs using such parameters. International Organization for Standardization (ISO) and the American National Standard Institute (ANSI) have developed standards, ISO1999 and ANSI S3.44, to predict the Hearing Threshold Levels (HTLs) at each frequency of 0.5, 1, 2, 3, 4 and 6 kHz for noise exposure levels between 75 and 110 dB for an exposure period of up to 40 years, for the ISO1999 standard, and 75 to 100 dB for the ANSI S3.44 standard. The results are of a statistical nature.

In parallel with the evolution of hearing threshold levels due to noise exposure, the aging factor which is present in all individuals is also taken into account. Both the ISO1999 and ANSI 83.44 standards have included the presbycusis factor based on the ISO7029 standard in their prediction formulas.

One standard approach used to check the effectiveness of hearing protectors is to test for an elevation of a person's hearing thresholds during or after noise exposure. An audiogram is normally administered by a trained professional who uses specialized audiometric equipment to test a person's hearing thresholds. This procedure requires space for the testing equipment in a quiet area to perform the hearing test. Current hearing testing devices also require the constant attention of a trained professional to test the hearing of the subject. This approach is time consuming.

To obviate the acquisition of ONIHL, the use of hearing protectors is mandatory for subjects exposed to 85, and sometimes 80 dB. The results of this procedure are evaluated in subjects with annual or periodic audiometric screening tests. In the US, these annual or periodic audiometric screening tests are associated with a mandatory evaluation of the Standard Threshold Shift (STS) as enacted by OSHA to determine if there is acquisition of ONIHL based on the evolution of HTLs at the 2, 3 and 4 kHz frequencies. The STS OSHA approach may indicate exposure to excessive noise levels.

ISO 7029 establishes a prediction of expected hearing loss in an individual as a function of age and gender. ISO1999 and ANSI S3.44 incorporate the ISO7029 standard in the prediction formulas in order to include the presbycusis factor to the effect of noise exposure to predict hearing loss that is likely to be experienced at each of the 6 different frequencies by an individual of a given age and gender exposed to a given noise level for a given period.

Based on such possible predictions, subjects exposed to potentially harmful noise exposure levels can be identified and measures can be taken to prevent potential hearing loss associated with continued estimated work conditions. As stated above, the most common recommendation is to have the worker wear an appropriate type of HPD if exposure to a noise level higher than 80 dB and is mandatory if the noise exposure is higher than 85 dB. Much research and development efforts are directed to the design and improvement of such HPD's.

Measuring the actual noise exposure of an individual wearing an HPD is practically impossible on a continued daily basis. Measures have been developed to evaluate for an individual the attenuation of an HPD whether they are muffs or insert HPDs. For example, ANSI developed two standards, with the acronyms MIRE (Microphone in Real Ear—used to insert HPDs) and REAT used for earmuffs. These procedures consist of evaluating the attenuation of selected insert HPDs specific to individual workers. For example, the 3M MIRE method measures actual attenuation by an insert HPD worn by a worker exposed to a controlled sound source. These procedures only apply to the day the test is performed. They do not take into consideration the manner in which the HPDs are worn, or whether they are worn continuously when the worker is exposed to noise on a daily basis. All of these procedures have a common factor: they are valid only for the day of the test. They do not take into consideration the duration the HPD was used or whether it was used properly for a specified period.

Companies are facing an increasing number of claims from workers seeking monetary compensation for their hearing loss condition that they associate as being caused by their work conditions. According to the Hearing Health Foundation, from 2000 to 2015 the number of Americans with hearing loss doubled. Unfortunately, companies have very weak arguments to demonstrate that the hearing loss incurred is not the sole effect of the working conditions. Large sums of money are paid in compensation and ancillary costs. Other conditions, of intrinsic or extrinsic nature, not being work related, could have led to a given degree of hearing loss or abnormal pattern for an individual. Social habits (e.g. non-occupational exposure to noise, smoking), medical conditions (e.g. genetics, trauma, ototoxic medication, infection), improper wearing of the recommended HPD, time an HPD is worn, are also amongst possibilities that could have contributed to the degree of hearing loss. There is therefore a need to better understand the etiology of hearing loss. Companies are thus interested in finding arguments to demonstrate the effectiveness of the HPDs worn by the individual.

Although standards such as ISO1999 and ANSI S3.44 enable predicting hearing loss that would likely be caused to a group of individuals being exposed to a given noise level, there is no known methodology to determine what conditions led to a given evolution from an initial audiometric report to a second or serial audiometric reports spaced by known periods for a specific individual.

Therefore, it would be a very significant advance in the field of hearing loss prevention if one could identify more readily problematic subjects by estimating the real noise exposure levels that led an observed hearing loss evolution. For example, one could determine that a given degradation of hearing condition at specific frequencies in a specific individual is likely to be caused by an exposure to an high noise level over a selected period while the correct and responsive wearing of the HPD would have normally reduced the estimated noise level to levels below 85 dB or less as perceived at the employee's ears.

It would be of great use, if for specific periods, with or without the use of a known HPD, the estimated noise level exposure at each frequency could be compared to the attenuation provided by the specific HPD as compared to the laboratory attenuation based on either the NRR procedure legislated by OSHA or the High-Medium-Low (HML) approach of ISO.

ISO1999 and ANSI S3.44 predict different hearing loss at each frequency; there is a greater hearing loss noted in the high frequencies of 3, 4 and 6 kHz as compared to 0.5, 1 and 2 kHz. When the observed relationship does not conform to these predictions at a frequency or series of frequencies; i.e., an abnormal relationship of low, mid, or high frequencies, such variation can lead to abnormal estimated noise levels. Such variations can be attributed to etiologies other than noise exposure.

Although standards such as ISO1999 and ANSI S3.44 enable predicting hearing loss that would likely be caused to an individual being exposed to a given noise level, there is no known methodology to determine what conditions led to a given evolution from an initial audiometric report to a second audiometric or serial reports spaced by known periods for a specific individual. There is therefore a need for improved methods and systems for inferring noise level exposure that was likely experienced by an individual for causing an observed degradation of hearing acuity.

### SUMMARY

The procedure proposed is based on the fact that if the age, gender, duration of exposure and evolution of HTLs at the 0.5, 1, 2, 3, 4 and 6 kHz are known for an individual, it is possible to determine the noise exposure level that would have resulted in the observed evolution of HTLs at each frequency and globally.

A specific individual may be classified as otologically normal for a certain period. However, the procedure allows identification of abnormal frequency variation that may be subjective of an intrinsic and/or extrinsic factors that may have worsened some or all of the HTLs in a matter not compatible with ONIHL and the aging factor.

A system of one or more computers can be configured to perform particular operations or actions by virtue of having software, firmware, hardware, or a combination of them installed on the system that, in operation, causes or cause the system to perform the actions. One or more computer programs can be configured to perform particular operations or actions by virtue of including instructions that, when executed by data processing apparatus, cause the apparatus to perform the actions. One general aspect includes a method for estimating a noise exposure level to which an individual having an age and a gender has been exposed during an exposure duration, an exposure to the noise exposure level having induced an evolution of hearing threshold value (HTL) of the individual, the method including: —obtaining a test audiogram of the individual measured at a time of testing; —obtaining a reference audiogram of the individual at a beginning of the exposure duration; —inputting the individual's gender, the individual's age at the time of testing, and the exposure duration at the time of testing in a prediction formula; —calculating, using the prediction formula and the reference audiogram, a plurality of projected audiograms each associated with a possible noise exposure level; —for each of the plurality of projected audiograms, comparing the projected audiograms with the test audiogram; —performing a curving fitting operation to select one audiogram, among the plurality of projected audiograms, that best fits the test audiogram; and—selecting the noise exposure level associated with the selected projected audiogram as an estimated noise exposure level to which the individual was exposed having induced the evolution of hearing threshold value (HTL) of the individual. Other embodiments of this aspect include corresponding computer systems, apparatus, and computer programs recorded on one or more computer storage devices, each configured to perform the actions of the methods.

Implementations may include one or more of the following features. The method where obtaining a reference audiogram includes producing a baseline audiogram and assigning the baseline audiogram to the reference audiogram, where the baseline audiogram corresponds to an audiogram of an otologically normal individual before exposure to the noise exposure level. The method where the obtaining a reference audiogram includes obtaining an initial audiogram of the individual by measuring at the beginning of the exposure duration to the noise exposure level and assigning the initial audiogram to the reference audiogram. The method where the initial audiogram is based on tests at a plurality of frequencies. The method further including: —producing a third audiogram of the individual by setting the HTL at each of the plurality of frequencies to a value specified in an aging factor table for a 0.5 percentile for each of the frequencies, for an individual of the individual's gender and the individual's age, thereby providing a third audiogram corresponding to a normal hearing acuity of an individual of the individual's gender and individual's age having a hearing loss solely affected by aging; —comparing the noise exposure levels of the selected audiogram with the HTLs of the third audiogram on a frequency by frequency basis; and—if two or more HTLs of the selected projected audiogram are higher than the HTL of the third audiogram at the same frequency as the initial audiogram to established invalid and that a valid noise exposure level could not be obtained from using the initial audiogram. The method where the initial audiogram provides a specific HTL for each of a plurality of frequencies, the method further including: —comparing each specific HTL with values provided in an aging factor table for the individual's gender and at the individual's age at the time of testing for percentile values ranging from 0.1 to 0.9 on a frequency by frequency basis; and—if two or more HTLs of the initial audiogram correspond to percentile values higher than 0.5 for the same frequencies, then considering the initial audiogram to be invalid and that a valid noise exposure level could not be obtained from using the initial audiogram. The method where the reference audiogram is tested by setting an HTL at each of a plurality of frequencies to a value specified in an aging factor table for the 0.5 percentile for each of the plurality of frequencies. The method further including obtaining the aging factor table from standard ISO7029. The method where setting a first noise exposure level to the estimated noise exposure level, where a second noise exposure level is selected based on a second audiogram tested by setting an HTL at each of a plurality of frequencies to a value specified in an aging factor table for the 0.5 percentile for each of the plurality of frequencies. The method may also include the method further including comparing the first noise exposure level to the second noise exposure level and selecting the highest of the first noise exposure level and the second noise exposure level as the estimated noise exposure level. The method further including obtaining the prediction formula from the ISO1999 or ANSI S3.44 standard. The method where the selected noise exposure level is selected from the group including level values ranging from 75 to 110 db. The method where selecting the projected audiogram includes using a statistical data fitting formula. The method where the statistical data fitting formula includes one of: a Smooth Huber Loss Function formula; a robust class of formulas; and a Smooth Huber Loss Function Robust formula. The system where the computer program is further for producing a baseline audiogram, where the baseline audiogram corresponds to an audiogram of an otologically normal individual before exposure to the noise exposure level. The system where the user interface is further for inputting an initial audiogram of the individual measured at a time before the exposure to the noise exposure level. The system further including: —a service provider server for receiving data inputted at the user interface, and—service provider computing facilities including the computer digital storage, the processing unit and the output device, where the service provider computing facilities further include communication means for transmitting the estimated noise exposure level to the user interface along with the information about the individual. Implementations of the described techniques may include hardware, a method or process, or computer software on a computer-accessible medium.

One general aspect includes a system for estimating a noise exposure level to which an individual having an age and a gender has been exposed during an exposure duration, an exposure to the noise exposure level having induced an evolution of hearing threshold value (HTL) of the individual, the system including: —a user interface for inputting a test audiogram of the individual measured at a time of testing, the individual's gender, the individual's age at the time of testing, the exposure duration at the time of testing, and identification of the individual; —a computer digital storage storing a computer program for performing calculation of an estimated noise exposure level deemed of having induced an evolution of hearing threshold value (HTL) of the individual over the exposure duration, where performing the calculation includes the steps of: —calculating, using a prediction formula and a reference audiogram of the individual at a beginning of the exposure duration, a plurality of projected audiograms each associated with a possible noise exposure level; —for each of the plurality of projected audiograms, comparing the projected audiograms with the test audiogram; —performing a curving fitting operation to select one audiogram, among the plurality of projected audiograms, that best fits the test audiogram; and—selecting a noise exposure level associated with the selected projected audiogram as the estimated noise exposure level to which the individual was exposed having induced an evolution of hearing threshold value (HTL) of the individual. The system also includes—a processing unit for executing the computer program. The system also includes—an output device for outputting information about the individual and the estimated noise exposure level. Other embodiments of this aspect include corresponding computer systems, apparatus, and computer programs recorded on one or more computer storage devices, each configured to perform the actions of the methods.

Implementations may include one or more of the following features. The system where the computer program is further for producing a baseline audiogram, where the baseline audiogram corresponds to an audiogram of an otologically normal individual before exposure to the noise exposure level. The system where the user interface is further for inputting an initial audiogram of the individual measured at a time before the exposure to the noise exposure level. The system further including: —a service provider server for receiving data inputted at the user interface, and—service provider computing facilities including the computer digital storage, the processing unit and the output device, where the service provider computing facilities further include communication means for transmitting the estimated noise exposure level to the user interface along with the information about the individual. Implementations of the described techniques may include hardware, a method or process, or computer software on a computer-accessible medium.

One general aspect includes a computer method for estimating a noise exposure level to which an individual having an age and a gender has been exposed during an exposure duration, an exposure to the noise exposure level having induced an evolution of hearing threshold value (HTL) of the individual, the method including: —providing to a user access to a computer application resident in a computer; —receiving from the user a test audiogram of the individual measured at a time of testing, the age of the individual at the time of testing, the exposure duration at the time of testing, and the gender of the individual; —using the computer application to perform the steps of: —calculating, using a prediction formula and a reference audiogram of the individual at a beginning of the exposure duration, a plurality of projected audiograms each associated with a possible noise exposure level; —for each of the plurality of projected audiograms, comparing the projected audiograms with the test audiogram; —performing a curving fitting operation to select one audiogram, among the plurality of projected audiograms, that best fits the test audiogram; and—selecting a noise exposure level associated with the selected projected audiogram as the estimated noise exposure level to which the individual was exposed having induced the evolution of hearing threshold value (HTL) of the individual. The computer method also includes—generating a report including information about the individual and the estimated noise exposure level. Other embodiments of this aspect include corresponding computer systems, apparatus, and computer programs recorded on one or more computer storage devices, each configured to perform the actions of the methods.

According to an aspect, the method further comprises: —producing a third audiogram of the individual by setting the HTL at each of the plurality of frequencies to a value specified in an aging factor table for a 0.5 percentile for each of the frequencies, for an individual of the individual's gender and the individual's age, thereby providing a third audiogram corresponding to a normal hearing acuity at a beginning of the exposure duration of an individual of the individual's gender and individual's age having a hearing loss solely affected by aging; —comparing the HTL of the selected projected audiogram with those of the third audiogram; and—if two or more HTLs of the selected projected audiogram are higher than the HTLs of the third audiogram on a frequency by frequency basis for two or more frequencies, then it is established that the initial audiogram is invalid and a valid noise exposure level could not be obtained from using the initial audiogram.

According to an aspect, the step of performing a curving fitting operation comprises identifying patterns in the projected audiograms and in the test audiogram and comparing the patterns.

According to an aspect, the method further comprises obtaining a plurality of periodic audiograms of the individual measured at a plurality of times during the exposure to the noise exposure level; and for each of the periodic audiograms, selecting the possible noise exposure level associated with the selected corresponding projected audiogram as the estimated noise exposure level to which the individual was exposed at the time of testing of the periodic audiogram.

According to an embodiment, there is described a method for determining a noise exposure level associated as the cause of an observed evolution of hearing acuity of an individual of known gender over an exposure period, the method comprising: —providing a first audiogram of the individual measured at a plurality of frequencies at age Y; —generating a second audiogram of the individual at age X, Y and X being integer values with Y being larger than X, said second audiogram being generated by setting a hearing threshold level at each of said plurality of frequencies to a value specified in an aging factor table for the 0.5 percentile for each of the frequencies, for an individual of same gender at age X, to provide an audiogram corresponding to the normal hearing acuity of an individual of that gender at age X having a hearing loss solely affected by aging; —inputting the individual's gender, age X, an exposure duration equal to Y−X in a statistical hearing threshold levels evolution prediction formula; —calculating projected hearing loss audiograms specific to each of a plurality of selected possible noise level exposure values, using the prediction formula; —comparing a pattern of each calculated projected audiogram with a pattern of the second audiogram; —selecting the projected audiogram that best fits the first audiogram; and—assuming that the noise exposure level value associated with the selected projected audiogram is the noise exposure level value that caused the evolution of hearing acuity observed between the second audiogram to the first audiogram.

According to an embodiment, there is described a method for determining a validated noise exposure level associated as the cause of an observed evolution of hearing acuity of an individual of known gender over an exposure period, the method comprising: —performing the method to determine a first noise exposure level value; —performing the method to determine a second noise exposure level value; and—comparing the first noise exposure value to the second noise exposure value: if the first noise exposure value is higher than the second noise exposure value, then the first noise exposure value is confirmed as the validated noise exposure value, else if the first noise exposure value is lower than the second noise exposure value, then the second noise exposure value is selected as the validated noise exposure value.

According to an embodiment, there is described a method for determining a validated noise exposure level associated as the cause of an observed evolution of hearing acuity of an individual of known gender over an exposure period, the method comprising: —providing a first audiogram of the individual measured at age X and a second audiogram of the individual measured at age Y, Y and X being integer values with Y being larger than X; —inputting the individual's gender, age X, an exposure duration equal to Y−X in a statistical hearing threshold levels evolution prediction formula; —calculating projected hearing loss audiograms specific to each of a plurality of selected possible noise level exposure values, using the prediction formula; —comparing a pattern of each calculated projected audiogram with a pattern of the second audiogram; —selecting the projected audiogram that best fits the second audiogram, said projected audiogram having a hearing threshold level value corresponding to each a of plurality of frequencies; —assuming that the noise exposure level value associated with the selected projected audiogram is the noise exposure level value that caused the evolution of hearing acuity observed between the first and the second audiograms; —generating a third audiogram of the individual at age X, said third audiogram being generated by setting a hearing threshold level at each of said plurality of frequencies to a value specified in an aging factor table for the 0.5 percentile for each of the frequencies, for an individual of same gender at age X, to provide an audiogram corresponding to the normal hearing acuity of an individual of that gender at age X having a hearing loss solely affected by aging; and—comparing the hearing threshold level values of the selected audiogram with those of the third audiogram on a frequency by frequency basis: if the hearing threshold level value of the selected audiogram at a frequency is higher than the hearing threshold level value of the third audiogram at the same frequency for two frequencies or more, then the noise exposure level value is rejected and the first audiogram is considered invalid, else the noise exposure level value is considered valid.

According to an embodiment, there is described a method for determining a validated noise exposure level associated as the cause of an observed evolution of hearing acuity of an individual of known gender over an exposure period, the method comprising: —providing a first audiogram of the individual measured at age Y, Y being an integer value, said audiogram providing a specific hearing threshold level value for each of a plurality of frequencies; —comparing each specific hearing threshold value with the values provided in an aging factor table for the given gender at age Y for percentile values ranging from 0.1 to 0.9 on a frequency by frequency basis: if two or more threshold level values of the first audiogram correspond to percentile values higher than 0.5 for the same frequencies, then the first audiogram is considered invalid and a valid noise exposure level could not be obtained from that audiogram; —else, a second audiogram of the individual is provided, measured at age X, X being an integer value and Y being larger than X; —inputting the individual's gender, age X, an exposure duration equal to Y−X in a statistical hearing threshold levels evolution prediction formula; —calculating projected hearing loss audiograms specific to each of a plurality of selected possible noise level exposure values, using the prediction formula; —comparing a pattern of each calculated projected audiogram with a pattern of the second audiogram; —selecting the projected audiogram that best fits the second audiogram, said projected audiogram having a hearing threshold level value corresponding to each a of plurality of frequencies; and—assuming that the noise exposure level value associated with the selected projected audiogram is the validated noise exposure level value that caused the evolution of hearing acuity observed between the second audiogram and the first audiogram.

According to an aspect, the aging factor table is provided from standard ISO7029.

According to an aspect, the prediction formula is obtained from the ISO1999 or the ANSI S3.44 standard.

According to an embodiment, there is provided a method for determining an estimated noise exposure level associated as the cause of an observed hearing acuity evolution in an individual of known gender, over an exposure period. The method comprises: 1) providing a first audiogram of the individual measured at age X and a second audiogram of the individual measured at age Y. Y and X being integer and Y being larger than X; 2) inputting the individual's gender, age X, an exposure duration equal to Y−X in a statistical hearing threshold levels evolution prediction formula and the first audiogram; 3) calculating projected hearing loss audiograms specific to each of a plurality of possible noise level exposure values, using the prediction formula: 4) comparing a pattern of each calculated projected audiogram with a pattern of the second audiogram; 5) selecting the projected audiogram that best fits the pattern of the second audiogram; 6) assuming that the noise exposure level value associated with the selected projected audiogram is the noise exposure value that caused the evolution of hearing acuity observed between the first and the second audiogram.

According to an embodiment, the prediction formula is obtained from the ISO1999 or ANSI S3.44 standards.

According to a further embodiment, selecting the projected audiogram comprises using a statistical data fitting formula. A statistical formula of the robust class may be used therefor, such as the Smooth Huber Loss Function Robust formula (SHLFR).

According to another aspect of the disclosure, there is provided a system for estimating a noise exposure level associated as the cause of an observed evolution of hearing acuity of an individual of known gender over a period. The system comprises: a user interface for inputting a first audiogram of the individual measured at age X and a second audiogram of the individual measured at age Y, value of X, value of Y or Y−X, the gender, and identification information of the individual; computer digital storage storing a computer program for performing calculation of a noise exposure level value deemed to have caused evolution of the individual's hearing acuity from age X to age Y; a processing unit for executing the program and an output device for outputting information about the individual and the noise exposure level value.

According to an embodiment, the system may further comprise a service provider server for receiving data inputted at the user interface, and service provider computing facilities comprising the processing unit and the computer digital storage. The provider computing facilities may further comprise communication means for transmitting the noise exposure level value to a user along with the information about the individual.

According to a further aspect, the present disclosure provides a method for estimating a noise exposure level associated as the cause of an observed evolution of hearing acuity of an individual of known gender over an exposure period. The method comprises: 1) receiving from a client a first audiogram of the individual measured at age X and a second audiogram of the individual measured at age Y, value of X, value of Y and the gender and identification information of the individual; 2) Performing calculation of the estimated noise exposure level value using a computing facility; and 3) sending a report to the client, the report comprising information about the individual and the calculated estimated noise exposure level value.

According to a still further aspect, there is provided a method for enabling a user to estimate a noise exposure level associated as the cause of an observed evolution of hearing acuity of an individual of known gender over an exposure period. The method comprises: 1) accessing a computer application resident in a computer under the user's control; 2) inputting in the application a first audiogram of the individual measured at age X and a second audiogram of the individual measured at age Y, value of X, value of Y and the gender of the individual; 3) using the application to perform calculation of the estimated noise exposure level value; and 4) generating a report comprising information about the individual and the calculated estimated noise exposure level value.

### BRIEF DESCRIPTION OF THE DRAWINGS

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature and not as restrictive and the full scope of the subject matter is set forth in the claims.

It will be noted that throughout the appended drawings, like features are identified by like reference numerals.

FIG. 1 is a sample series of audiograms for an individual;

FIG. 2 is a flowchart for executing a method according to an embodiment of the present disclosure;

FIG. 3 is a flowchart for interpreting a real ear exposure level (REEL) and determining if it could have caused the evolution of HTLs of an individual from an Audiogram 1 to an Audiogram 2;

FIG. 4 is a visual comparison between three projected audiograms fitted with three different fitting algorithms and a sample Audiogram 2;

FIG. 5 is a table of REEL results using lower and upper bound values from a sample Audiogram 2 as well as an audiogram with a modified HTL value at 500 Hz serving as an outlier to test the fitting algorithms;

FIG. 6 shows a comparison between ISO1999 and ANSI S3.44 HTL projections for a 21 year old male exposed to 90 dBA during various exposure durations and a sample audiogram with a non-noise induced HTL evolution;

FIG. 7 is an example of a summary table of results for REEL values for a male exposed to noise between ages 21 and 60;

FIG. 8 is an example of a table of REEL results calculated for each frequency;

FIG. 9 is a table of sample earplug attenuations for each frequency;

FIG. 10 is an example of a report produced according to a step of the method of FIG. 2;

FIG. 11 is an example of an administrative report, according to a step of the method of FIG. 2, demonstrating a matrix with each case corresponding to the number of individuals tested that correlates the REEL value of an individual with the noise level at the individual's workstation;

FIG. 12 shows the a coefficient table to be used in connection with a formula of the ISO1999 standard;

FIG. 13 is an example of the table of results for presbycusis predictions for individual of age 60, according to the ISO7029 standard, Annex A;

FIG. 14 is an example of the table of Noise Induced Permanent Threshold Shift (NIPTS) results;

FIG. 15 is an example of the table of results for hearing loss values according to ISO1999 and the projected audiogram;

FIG. 16 is an example of the projected audiogram of a 60-year old male with 39 years of exposure to 90 dB according to ISO1999 projection formula;

FIG. 17 is an example of the table of distances between a sample ISO1999 projected audiogram and a sample Audiogram 2;

FIG. 18 is an example of the Smooth Huber Loss Function Robust distances between an ISO1999 projected audiogram and an Audiogram 2;

FIG. 19 is a superposition of a sample Audiogram 1 and sample Audiogram 2 with a sample projected audiogram with the best fit to Audiogram 2 according to Smooth Huber Loss Function Robust;

FIG. 20 is an example of the table of hearing loss projections according to ISO1999 for a male exposed to 90 dB between ages 21 and 62;

FIG. 21 summarizes the table of constants Xu, Yu, XI, and YI for ISO1999 percentile projections of hearing loss;

FIG. 22 is an example of the table of results for ISO1999 percentile calculations;

FIG. 23 is an example of hearing loss projections according to 10% and 90% percentile for a 60-year old male exposed to 90 dB from age 21 to 60;

FIG. 24 is a flowchart of method for determining a REEL associated as a cause of an evolution of hearing acuity of an individual, using a service provider's WEB server connection for remote calculations, according to an embodiment;

FIG. 25 is a schematic representation of a system wherein a server computer accessible via Internet may receive data from a local computer and send back corresponding REEL values and interpretation;

FIG. 26 is a flowchart of a method for estimating a REEL as a cause of an evolution of hearing acuity of an individual, according to an embodiment;

FIG. 27 is a schematic representation of a system using a local computer to compute REEL values, according to an embodiment; and

FIG. 28 is a presbycusis defined as the evolution of HTLs of an otologically normal subject as defined by ISO7029 with respect to the age and gender of the subject.

### DEFINITIONS

For purposes of this description, within the context of this specification, each term or phrase below includes the following meaning or meanings.

Audiogram means result of an audiometric test under graphical or table form, indicating hearing threshold levels in decibels in an individual's ear for each of a plurality of measured frequencies. Usually, six standardized frequencies are considered: 0.5, 1, 2, 3, 4, and 6 kHz.

Audiogram 1 is the audiogram that is used as a baseline at the beginning of the period upon which the HTL evolution is analyzed. It is also referred to as the baseline audiogram.

Audiogram 2 is a second audiogram of the same individual at the end of the period which is analyzed. Serial audiograms are audiograms performed on an annual or periodic basis in a hearing conservation program.

The projected audiogram is the audiogram obtained by using the ISO1.999, or ANSI S3.44 projection formulas.

HL_{50%}¹⁹⁹⁹ is the projected hearing threshold level with varying age, gender, noise exposure level, and noise exposure duration according to ISO1999 and ANSI S3.44.

HL_{50%}⁷⁰²⁹ is the projected hearing threshold level of an individual according to ISO7029 using the age and gender of the individual.

HL_{50%, age}⁷⁰²⁹ is the hearing threshold level for the median population according to ISO7029 using the age and gender of the individual for Audiogram 2.

HL_{50%, Baseline Age}⁷⁰²⁹ is the hearing threshold level for the median population according to ISO7029 using the age and gender of an individual for Audiogram 1 corresponding to baseline.

NIPTS_{50%}, is the noise-induced permanent threshold shift obtained by the following ISO1999 and ANSI S3.44 formula:

$${HL}^{1999} = {HL}^{7029} + NIPTS - \frac{{HL}^{7029} + NIPTS}{120} .$$

HL_{baseline} is the hearing threshold level of an individual as measured by the hearing test results in Audiogram 1 corresponding to baseline; i.e., beginning of the period analyzed.

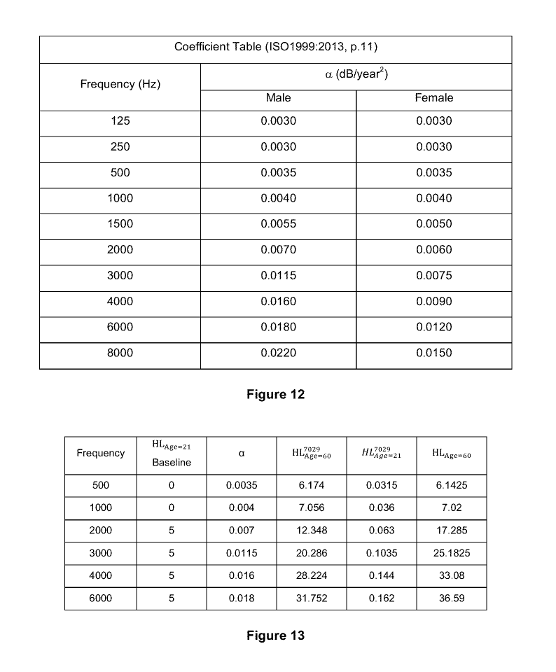

α is the constant from tables in ISO1999, depending on gender (FIG. 12).

Lex,8 h is the noise exposure level normalized to a nominal 8 h working day.

Lo is the sound level, below which the effect on hearing is negligible (ISO1999 table, p. 6).

t is the exposure duration, expressed in years.

to is always set to 1 year.

u and v are constants given as a function of frequency in ISO1999.

Baseline age is the age of the individual for Audiogram 1.

Presbycusis is defined as the evolution of HTLs of an otologically normal subject as defined by ISO7029, Annex A with respect to the age and gender of the subject.

Extrinsic factors include noise, trauma, ototoxic drugs, barotraumas, explosions, ototoxic environment.

Intrinsic factors include genetics, presbycusis, otologic and systemic diseases.

Curve fitting is the process of constructing a curve or mathematical function that has the best fit to a series of data points (forming a data curve), possibly subject to constraints. Curve fitting can involve either interpolation, where an exact fit to the data is required, or smoothing, in which a “smooth” function is constructed that approximately fits the data. A related topic is regression analysis, which focuses more on questions of statistical inference such as how much uncertainty is present in a curve that is fit to data observed with random errors. Fitted curves can be used as an aid for data visualization, to infer values of a function where no data are available, and to summarize the relationships among two or more variables. Extrapolation refers to the use of a fitted curve beyond the range of the observed data, and is subject to a degree of uncertainty since it may reflect the method used to construct the curve as much as it reflects the observed data.

In the following description, for purposes of explanation and not limitation, specific details are set forth such as particular architectures or techniques. It will be apparent to those skilled in the art that the system and methods described hereinafter may be practiced in other embodiments that depart from these specific details.

### DETAILED DESCRIPTION

In embodiments, there are disclosed methods and systems for determining an estimated noise exposure level responsible of an observed evolution of hearing acuity of an individual of known gender and age over a known period. Methods are further concerned with analysis of differences between measured and predicted hearing threshold levels.

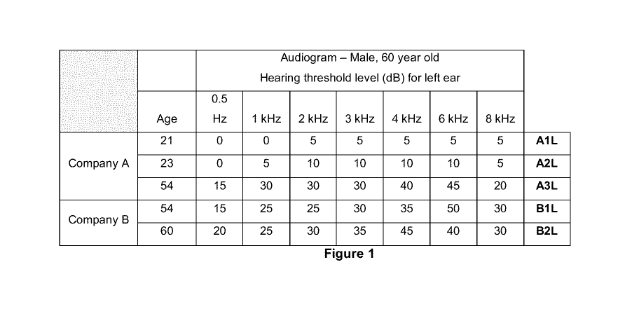

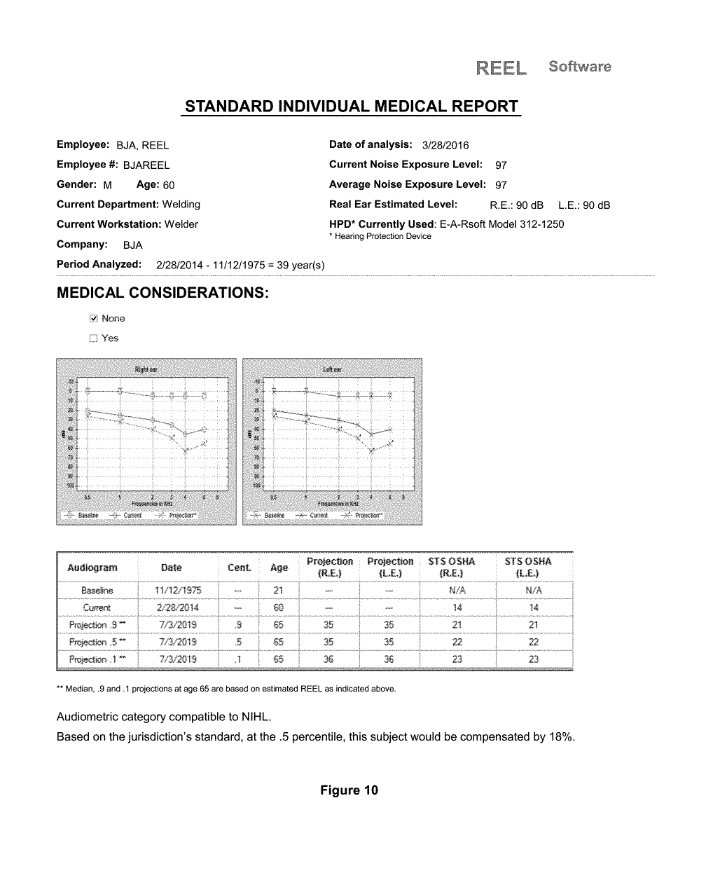

Referring more particularly to FIG. 1, there is shown a table of audiometric test results comprising a first series of 3 audiograms of an individual's left ear taken at company A at various ages between 21 (A1L) to 54 (A3L) years and a second series of 2 audiograms of the same individual between age 54 (B1L) and age 60 (B2L). Each audiogram comprises 7 values, each value indicating a hearing threshold level (HTL) in dB corresponding to an emitted acoustic frequency in Hz. The tests must be performed according to well defined procedures. The individual is positioned in a soundproof booth and wearing headphones is subjected to series of tones at different acoustic levels and frequencies and is asked to signal when each tone becomes audible. The lowest detected HTL for each frequency can be mapped in a table as in FIG. 1 or on a grid to form a series of dots (an example of which is seen in report such as FIG. 10).

The disclosed method uses as input separate audiograms: Audiogram 1 and Audiogram 2 from a same individual for each period analyzed in order to calculate the noise level that is deemed to have caused the progression of hearing loss between two audiograms based on a known prediction formula, such as those provided in standards ISO1999 or ANSI S3.44. This estimated noise level will be hereinafter referred to as the estimated real ear exposure level (REEL) value.

The disclosed method consists of evaluating the estimated noise exposure level for at least one specific period. This requires having baseline audiogram corresponding to the beginning of the period and a second audiogram at the end of the selected period. In the presence of serial audiograms analysis can be made for various periods in relation to either different noise exposure levels of different employers or work stations.

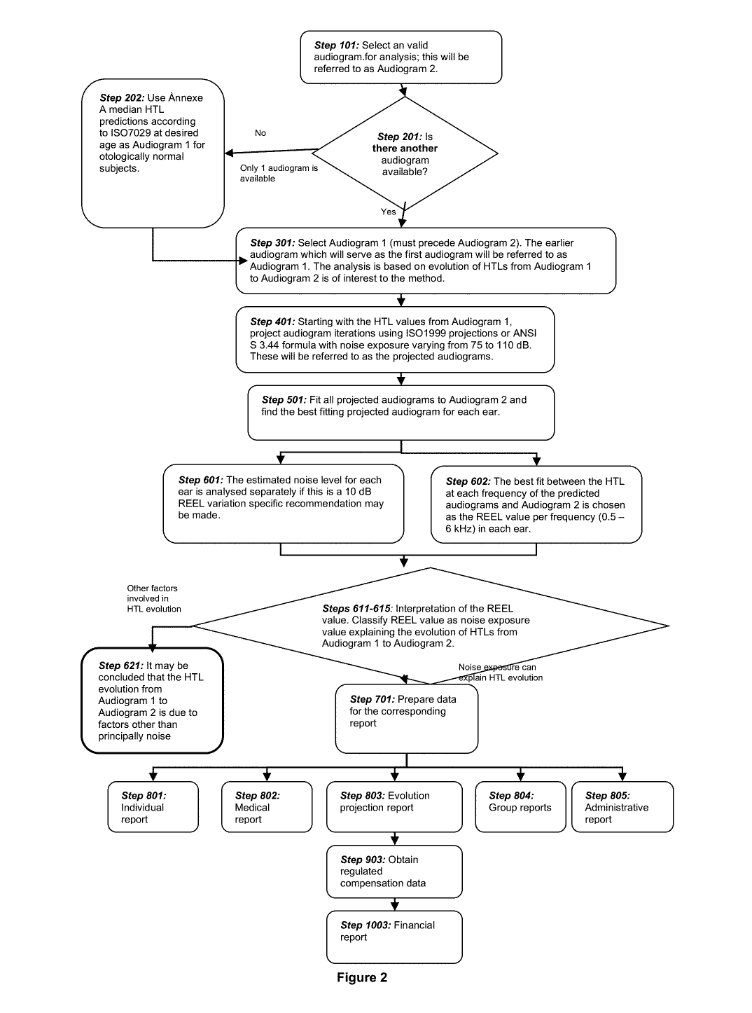

Referring to FIG. 2, a flowchart of a method according to an embodiment of the disclosure is shown. According to the first step 101 of the method, at least one valid audiogram obtained from audiometric screening tests of a specific individual as performed in HCP, audiogram B2L at age 60 for example, is selected for analysis and referred to as Audiogram 2.

To be valid, an audiogram must include the six frequencies of 0.5, 1, 2, 3, 4, and 6 kHz. An audiogram can be rejected if there is a difference of 50 dB or more between two consecutive frequencies.

Then, a test is performed at step 201: if a valid baseline audiogram prior to Audiogram A3L is available, for example audiogram A1L at age 21, it is selected as Audiogram 1 (Step 301).

One can observe that sensitivity values in the audiogram B2L are generally lower than those in audiogram A1L, indicating a loss of hearing acuity, particularly noticeable in the higher frequency region, which occurred during the time span between the two audiometric sessions. It is known that such a degradation of hearing acuity may in part result of aging as well as from exposure to noise.

If serial audiograms are available after performing an AQA, one is selected as the baseline, generally providing the broader lifetime gap (A1L) or the broader time gap at a given company (B1L) or work station. If there are more than 2 audiograms to choose from, it is recommended to pick the two with the largest time gap between them, typically the baseline, which is the first audiogram of an individual or simply the reference audiogram upon which the evolution of the individual's HTLs in time is evaluated, and the most recent valid audiogram. Typically, the larger the gap, the more significant the results over the course of the worker's career. However choosing the two audiograms with the largest time period is not a requirement. Although the noise level at the individual's workplace might change significantly over the years and in between each of the audiograms, the longer period between two audiograms ensures an average over the working period of that individual.

If at test 201 only one audiogram is available, it is selected as Audiogram 2. In this instance, for otologically normal subjects, a first (baseline) audiogram can be generated according to ISO7029, Annex A, 0.5 percentile projections for a person of the given gender of 18 years old or more, for example at the age of the first hiring date, (Step 202). This audiogram is selected as Audiogram 1 (Step 301) assuming the individual was otologically normal and had normal hearing at that age according to the 0.5 percentile of ISO7029. This may be useful in estimating the actual noise exposure of the individual with only a recent audiogram and with no prior hearing tests, but it lies on the assumption that the individual's hearing was similar to the ISO7029 predictions at the start of his career. Although not as accurate as having two audiograms, the method allows to estimate the noise exposure that would have caused the progression from normal hearing at a given age according to ISO7029 until the HTLs observed in the most recent audiogram.

The ISO1999 and ANSI S3.44 projection formulas were created to aid noise induced hearing loss risk managers, such as hygienists and health professionals, to evaluate the evolution of hearing threshold levels of an individual exposed to noise. In the following disclosure, it is proposed to use the HTLs of Audiogram 2 or serial audiograms as the result of an exposure to noise in order to estimate what noise level could have caused the evolution of those HTLs from Audiogram 1 to Audiogram 2 for that individual. Upon reviewing available literature, this method has not been applied by any other researcher in industry as the ISO1999 projection formulas were not created for this purpose. The disclosed method proposes a different innovative way of using such a projection formula in order to estimate REEL values that have been based on audiometric results of a specific individual rather than on noise measurements, such as dosimetry and sound level meter noise measurements. Audiograms used for the method must be valid and the audiometric tests must have been completed in compliance with the state of the art of hearing testing procedures in order to ensure the validity of the hearing tests. Once the validity of the tests has been established, then a pair of audiometric tests for a same individual may be used as Audiogram 1 and Audiogram 2 in the disclosed method.

As presented in FIG. 1 an audiogram from a typical audiometric test comprises 6 or more results per ear. The frequencies of 0.5, 1, 2, 3, 4, 6 kHz are mandatory.

From the results of the various tested frequencies from typical hearing tests, the method uses 6 frequencies from the audiogram for its analysis. The retained results are for frequencies 0.5, 1, 2, 3, 4, and 6 kHz as these are the ones that have predictions associated with them in the ISO1999 and ANSI S3.44 prediction formulas.

In ISO1999 and ANSI S3.44, the evolution of the HTL is given in various percentiles of the population ranging from 5% of the population to 95% and can be found in Annex A and Annex B of ISO1999 and ANSI S3.44.

For the purposes of ISO1999 and ANSI S3.44, the hearing threshold levels (in dB—indicated as HL in the formulas below) associated with age, gender, exposure duration, and noise exposure level of a noise-exposed population is calculated as follows, using the median values of the population for demonstration purposes:

$${HL}_{50 \%}^{1999} = {HL}_{50 \%}^{7029} + {NIPTS}_{50 \%} - \frac{{HL}_{50 \%}^{7029} * {NIPTS}_{50 \%}}{120} \\ {HL}_{50 \%}^{1999} = {HL}_{50 \%}^{7029} + {NIPTS}_{50 \%} - \frac{{HL}_{50 \%}^{7029} * {NIPTS}_{50 \%}}{120} If the exposure duration is between 10 years and 40 years , \\ {HL}_{50 \%}^{7029} = {HL}_{baseline} + {HL}_{50 \% , age}^{7029} - {HL}_{50 \% , Baseline Age}^{7029} \\ {HL}_{50 \%}^{7029} {HL}_{baseline} + α {( Age - 18 )}^{2} α {( Baseline Age - 18 )}^{2} \\ {HL}_{50 \%}^{7029} = {HL}_{baseline} + α {( Age - 18 )}^{2} - α {( Baseline Age - 18 )}^{2} {NIPTS}_{50 \% , t >= 10 , t <= 40} = [ u + v log ( \frac{t}{{t}_{0}} ) ] * {( {L}_{ex , 8 h} - {L}_{o} )}^{2} \qquad Formula 1$$

If the exposure duration is less than 10 years, we have

$${HL}_{50 \%}^{7029} = {HL}_{baseline} * \frac{{( Age - 18 )}^{2}}{{( Baseline Age - 18 )}^{2}}$$

$${HL}_{50 \%}^{7029} = {HL}_{baseline} * \frac{{( Age - 18 )}^{2}}{{( Baseline Age - 18 )}^{2}} {NIPTS}_{50 \% , t < 10} = \frac{log ( t + 1 )}{log ( 11 )} * {NIPTS}_{50 , t = 10}$$

If the exposure duration is greater than 40 years, then we set it to 40 years in the NIPTS term and proceed similarly for other terms in the ISO1999 (or ANSI 83.44) projection formula.

$${NIPTS}_{50 \% , t < 40} = [ u + v log ( \frac{40}{{t}_{0}} ) ] * {( {L}_{ex , 8 h} - {L}_{o} )}^{2}$$

The following three formulas for the S101999 (or ANSI S3.44) projections are obtained as a function of age, gender, exposure duration, and exposure level.

$$\begin{matrix}{HL}_{50 \% , t \geq 10 , t \leq 40}^{1999} = {HL}_{baseline} + α {( Age - 18 )}^{2} - α {( Baseline Age - 18 )}^{2} + [ u + v log ( \frac{t}{{t}_{0}} ) ] * {( {L}_{ex , 8 h} - {L}_{0} )}^{2} - α {( Age - 18 )}^{2} * [ u + v log ( \frac{t}{{t}_{0}} ) ] * {( {L}_{ex , 8 h} - {L}_{0} )}^{2} / 120 & Formula 2 \\ {HL}_{50 \% , t < 10}^{1999} = {HL}_{baseline} * \frac{{( Age - 18 )}^{2}}{{( Baseline Age - 18 )}^{2}} + \frac{log ( t + 1 )}{log ( 11 )} * [ u + v log ( \frac{10}{{t}_{0}} ) ] * {( {L}_{ex , 8 h} - {L}_{o} )}^{2} - \frac{\begin{matrix}α {( Age - 18 )}^{2} * \\ \frac{log ( t + 1 )}{log ( 11 )} * [ u + v log ( \frac{10}{{t}_{0}} ) ] * {( {L}_{ex , 8 h} - {L}_{0} )}^{2}\end{matrix}}{120} & Formula 3\end{matrix}$$

The analysis can be performed for company A from age 21 to 54 resulting in a certain real exposure value (REEL) for that period and for company B from age 54 to 60 giving a different REEL value.

$${HL}_{50 \% , t > 40}^{1999} = {HL}_{baseline} * α {( Age - 18 )}^{2} - α {( Baseline Age - 18 )}^{2} + [ u + v log ( \frac{40}{{t}_{0}} ) ] * {( {L}_{ex , 8 h} - {L}_{0} )}^{2} - α {( Age - 18 )}^{2} * [ u + v log ( \frac{40}{{t}_{0}} ) ] * {( {L}_{ex , 8 h} - {L}_{0} )}^{2} / 120 \\ At step 401 , {HL}_{50 \% , t > 40}^{1999} = {HL}_{baseline} * α {( Age - 18 )}^{2} - α {( Baseline Age - 18 )}^{2} + [ u + v log ( \frac{40}{{t}_{0}} ) ] * {( {L}_{ex , 8 h} - {L}_{0} )}^{2} - α {( Age - 18 )}^{2} * [ u + v log ( \frac{40}{{t}_{0}} ) ] * {( {L}_{ex , 8 h} - {L}_{0} )}^{2} - 120 \qquad Formula 4$$

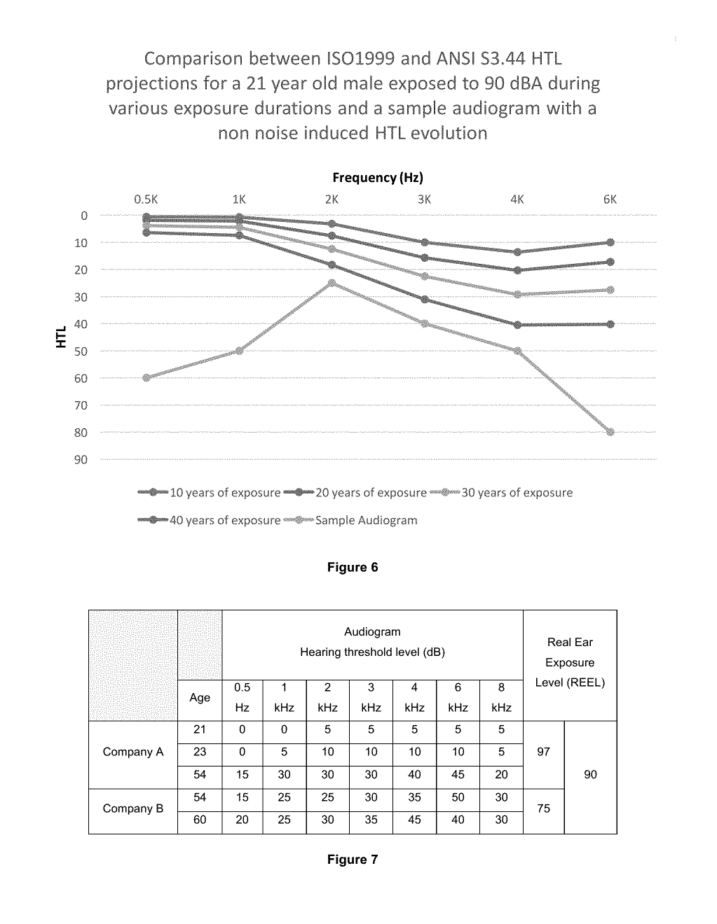

using Audiogram 1 and the number of years between Audiogram 1 and Audiogram 2 as the duration, as well as the age and gender of the individual, iterations are performed for noise levels (Lex, 8 h) ranging from 75 dB to 110 dB using either of the ISO1999 prediction formulas, i.e, one of Formula 2, Formula 3 or Formula 4 (as shown in FIG. 6 comparing ISO1999 predictions for a 60 year old male with an exposure duration of 39 years to an exposure level of 90 dB using Formula 3). The ANSI S3.44 formula has the same characteristics of the ISO1999 projection formulas; however it ranges for noise levels between 75 dB to 100 dB. The projected hearing loss values are added to the existing HTLs in Audiogram 2 for each of the exposure level iterations. Therefore 36 different predicted audiograms are obtained by applying the ISO1999 or 26 by applying the ANSI S3.44 formula hearing loss predictions for each frequency and noise level which will then be fitted with Audiogram 2.

In parallel to the ISO1999 and ANSI S3.44 procedures, the relationship of HTL to the prediction of ISO7029 is performed. The relationship of audiogram to the predictions of ISO7029 may modify the REEL levels in relation to the consideration that there may be for a certain period, independent of the evolution of HTLs, the HTLs may be compatible to the median of better percentiles of the ISO7029 predictions. In such cases, it could be concluded that the observed HTLs of audiogram 2 or several audiograms do not present any ONIHL. The estimated ONIHL is then determined to be 75 dB or less.

Step 501 the matching process of the calculated iterated ISO1999 or ANSI S3.44 projected audiograms with Audiogram 2 is done using statistical fitting tests. Mathematically, any fitting algorithm may be used to estimate the best fit between a given iteration projected HTL at each Hz and Audiogram 2.

If Audiogram 1 evolves to Audiogram 2 exactly along the predictions of the projection formula such ISO1999 or ANSI S3.44, regardless of the fitting method used, the estimated REEL value would always be deterministic and the same. However, in practice, discrepancies between the prediction formulas and observed HTLs are very common.

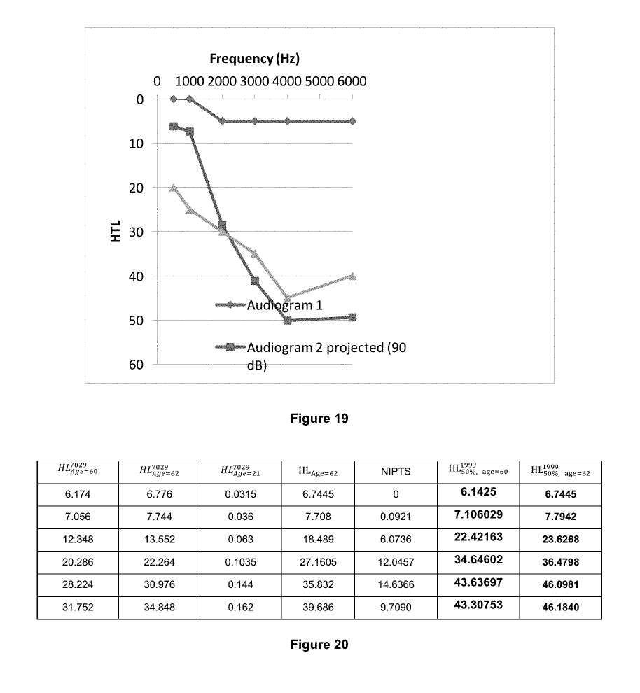

Audiograms that do not evolve with the same shape as the ISO1999 or ANSI S3.44 predictions are more prone to problems since fitting may be an issue. A solid statistical algorithm is used to fit the many HTLs from the projected audiogram to those of Audiogram 2 and find the best fitting projected levels. The statistical analysis was applied to several thousands of audiograms for different male and female individuals taken at different ages with known noise exposure conditions over various time spans. Hearing acuity monitoring in industry has been performed for many years. It was found that the statistical distributions that best fit the data were those included in the class of robust statistics. More specifically, the Smooth Huber Loss Function Robust (SHLFR) to evaluate the difference between the estimated audiogram with the projection formula of ISO1999, ANSI S3.44, ISO7029 and the actual audiogram was the best at handling various different audiogram output and audiogram matching problems. Problems mostly refer to outliers in audiometric data that cause inconsistencies with the general shape of the audiogram FIG. 19.

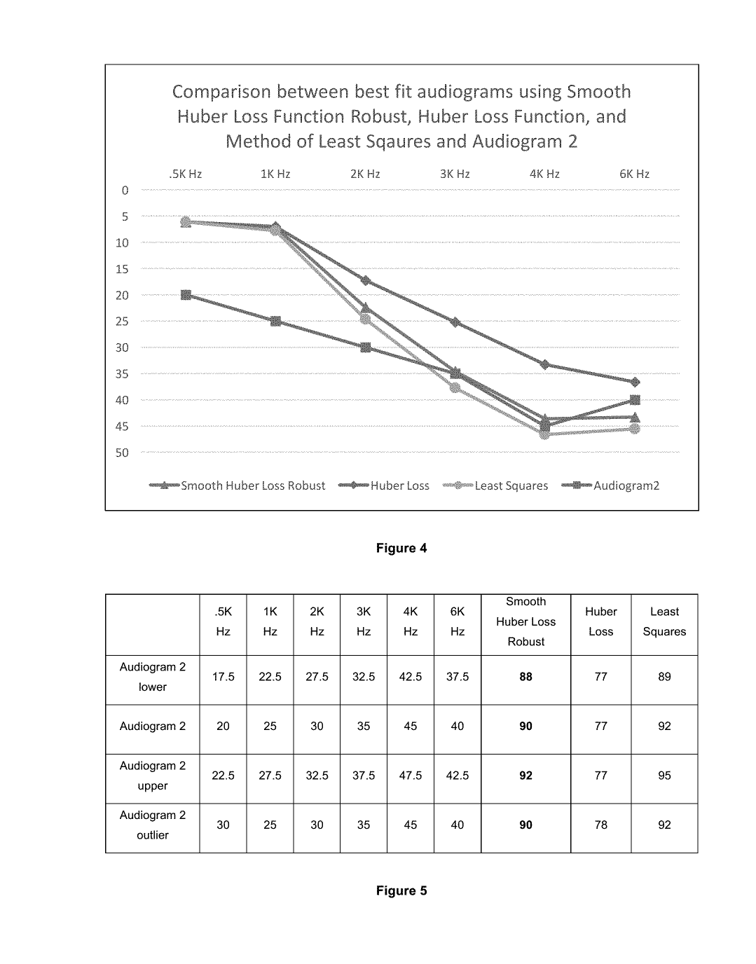

Three different fitting algorithms were used to estimate the REEL value that would be obtained by analyzing audiograms of A1L (company A) and B2L (company B) from FIG. 1; they are the Smooth Huber Loss Function Robust, the Huber Loss Function and the Method of Least Squares. Each fitting method found a projected audiogram of 90 dB, 77 dB, and 92 dB respectively to be the best fit. The results can be seen in FIG. 4. From FIG. 4, the result from the Huber Loss Function is rejected as it does not offer a good fit to Audiogram 2. For a health professional evaluating the audiograms and the REEL value (see Step 614 in FIG. 6)), this visual check is an important point amongst others in evaluating the REEL value.

The audiometric tests measure HTLs in steps of 5 dB, and therefore all actual HTLs between 17.5 dB and 22.49 dB will result in an HTL 20 dB. In FIG. 5, the lower and upper bounds of the HTL values from Audiogram 2 of Example 1 have been calculated. Using each of the three audiograms as Audiogram 2 in the proposed method, various REEL values were obtained.

From FIG. 5, the Smooth Huber Loss Function Robust has a tolerance of 2 dB and the Method of Least Squares has tolerance of 3 dB. Therefore, the SHLFR fitting algorithm yielded the best range of results for the REEL value.

From a statistical distribution point of view, some audiograms may have HTL values that are considered outliers in some frequencies. However, when robust statistics are applied, the estimated REEL should not be impacted by outliers. FIG. 5 also shows an example in which the 0.5 kHz HTL evolved from 20 dB to 30 dB, thus making it an outlier. The REEL value obtained with SHLFR did not vary from the initial audiogram and it is generally insensitive to variations of 10 dB or less. As described in [155], variations of 10 dB at a tested frequency may be inherent to the testing method. A statistical fitting algorithm that is insensitive to such a change in HTLs is considered an optimal fitting algorithm for audiograms.

It would be impossible to identify a statistical method which would cover all scenarios of HTL variations in audiograms. However, based on the number of cases analyzed, SHLFR seems to yield the most accurate results.

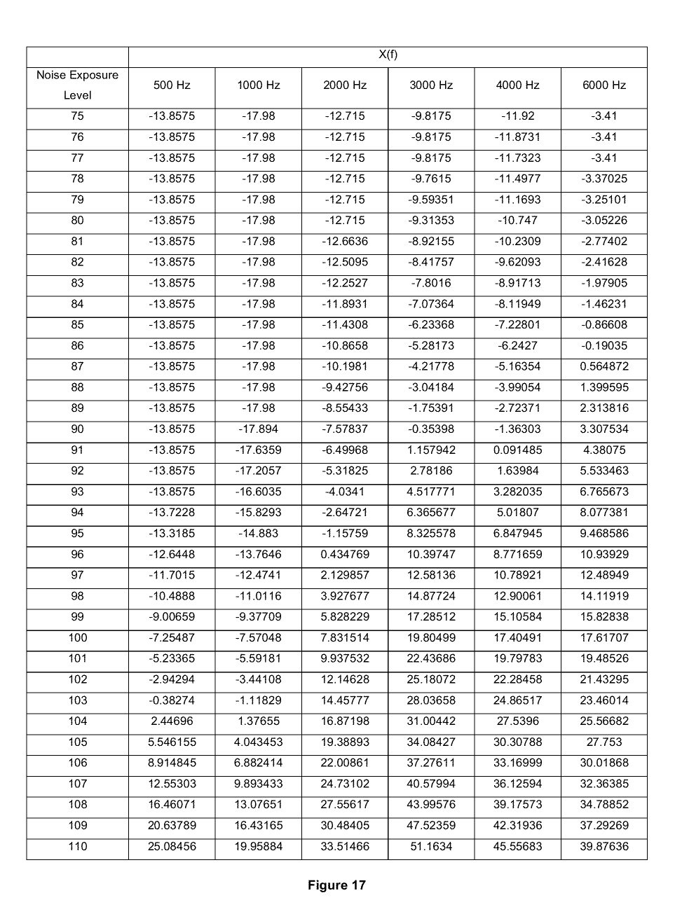

In order to establish the best fitting projected audiogram to Audiogram 2 of the individual, the difference between HTL values is calculated for each of the 6 frequencies between both these audiograms (an example of which is seen in FIG. 17). This calculation may be performed using weighting factors. Formula 5 (FIG. 17)

X(f)=d(f)=Projected HL¹⁹⁹⁹(f)−Audiogram 2(f)

SHLFR then uses this value to calculate a modified set of distances per frequency according to the following formula where a=3/2, b=1, and c=5/4.

$${X}^{′} ( f ) = \{ \begin{matrix}- c * X ( f ) , & X ( f ) \leq - a \\ \frac{{[ X ( f ) + a ]}^{3}}{3} - c * X ( f ) , & - a < X ( f ) < - b \\ \frac{[ {X}^{2} ( f ) - {b}^{2} ]}{2} + \frac{{( a - b )}^{3} + 3 bc}{3} , & - b \leq X ( f ) \leq b \\ - \frac{{( X ( f ) - a )}^{2}}{3} + c * X ( f ) , & b < X ( f ) < a \\ c * X ( f ) , & otherwise\end{matrix} \qquad Formula 6$$

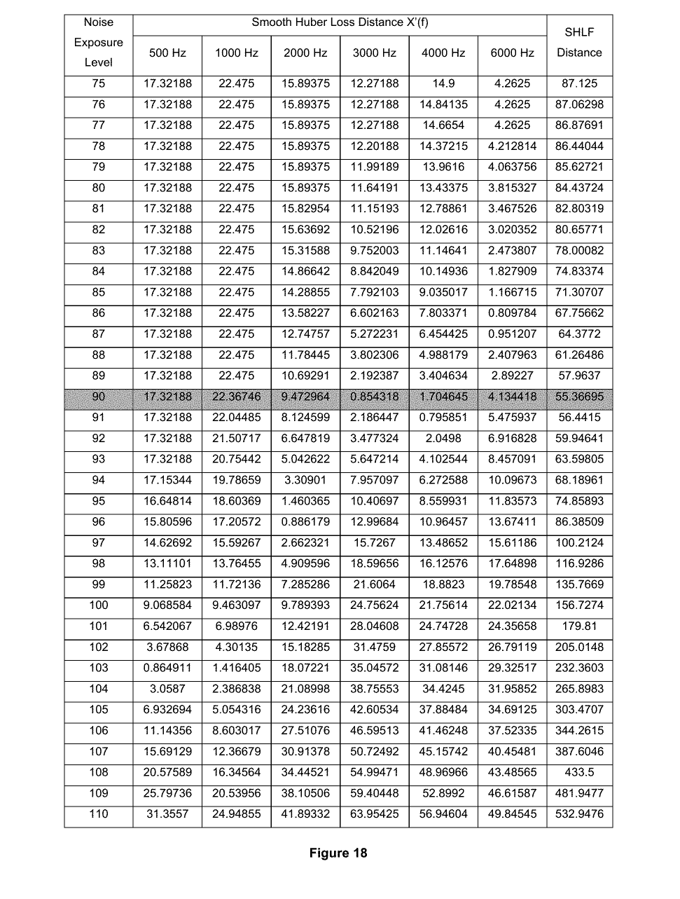

Once a table such as the one in FIG. 18 is obtained, the noise level whose sum of X′(f) at each frequency is the minimum is chosen as the noise level which generated a projected audiogram that best fits Audiogram 2 such as the one with the minimal distance in FIG. 18 (i.e. 90 dB).

Once the best fitting audiogram to the Audiogram 2 is established, the noise level which generated the iterated audiogram is chosen as the real ear noise exposure for that ear (Step 601). This noise level is said to be the actual noise exposure of the individual throughout the time between both the initial audiogram and the reference audiogram. This estimated noise level is herein referred to as the REEL.

The process may be repeated for each ear using HTL values from hearing tests similar to Audiogram 1 and Audiogram 2. An example of Step 401, Step 501, and Step 601 will be presented in Example 1 hereinafter.

Although Example 1 uses the median values of the population, the ISO1999, ANSI S3.44 and ISO7029 formulas give estimations for various percentiles ranging from 5% to 95% in regards to hearing loss projections. Percentiles for Noise Induced Permanent Threshold Shift (NIPTS) are obtained by using the NIPTS of the median and adding a correction factor it. A detailed example of this process is provided in Example 3.

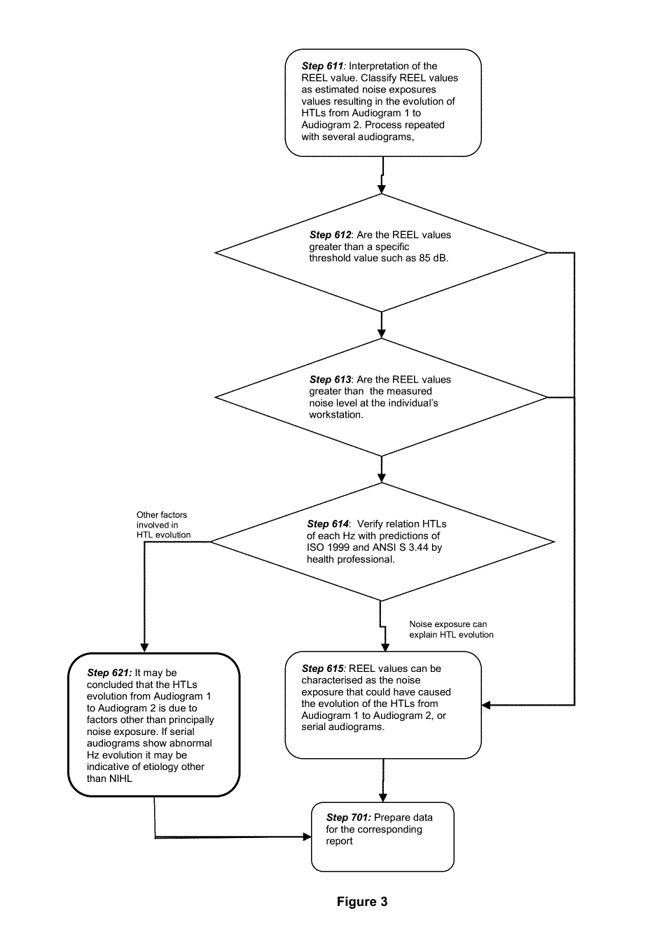

At Step 611, the REEL value in interpreted. The evaluation may proceed according to Step 611 to Step 615 detailed with reference to FIG. 3.

From Steps 611 to 614, if the REEL result is deemed to not have caused the evolution of HTLs from Audiogram 1 to Audiogram 2, then the evolution of the individual's HTLs may be due to factors not related to noise (Step 621). Otherwise, the REEL value is accepted (Step 615) and the REEL results are used in Step 701 along with complementary data for the preparation of different possible forms of reports. Examples of results are presented in FIG. 7.

Steps 611 to 615 in FIG. 3 are involved in the interpretation of the REEL result. In Step 612, as a general rule, any REEL results below a defined threshold are accepted as being the reason for HTL evolution from Audiogram 1 to Audiogram 2. The 80 dB or 85 dB threshold is commonly used in occupational hearing health risk management since a noise exposure below the 85 dB threshold has a low risk of causing hearing loss. A typical noise level threshold would also be one below which no executive action on the individual would be required, such as a follow up of his work habits or evaluation of the HPDs used. Then, the REEL value is accepted at Step 615 as having caused evolution of HTLs from Audiogram 1 to Audiogram 2, and the method proceeds with the reporting steps starting at Step 701.

In Step 613, should the REEL value of the individual be higher than 85 dB, then it would be compared with the measured ambient noise exposure level (NEL). Such an NEL reference value is often available from the individual's employer. Otherwise, it can be estimated from data published in public records in the literature in reference to many types of generic job functions and conditions. If the REEL value is below the NEL of the individual's workstation, then the evolution of the HTLs from Audiogram 1 to Audiogram 2 can be explained by a noise exposure equivalent to the REEL. The difference may be attributed to HPD attenuation or other factors such as the varying amount of exposure time and noise level. The REEL value is accepted at Step 615 as the deemed cause of the evolution of HTLs from Audiogram 1 to Audiogram 2, and the method proceeds with the reporting steps starting at Step 701.

If the REEL value is above the measured noise level of the individual's workstation, other factors of intrinsic or extrinsic origin that would have led to the observed HTL evolution from Audiogram 1 to Audiogram 2 must be evaluated by qualified persons such as a health professional or industrial hygienist (Step 614). In Step 614, the health professional would compare the REEL value with the evolution of the HTL patterns from Audiogram 1 to Audiogram 2 and validate the REEL value relative to the predicted evolution due to noise, presbycusis and both intrinsic and extrinsic factors.

There is a specific audiometric pattern for hearing loss due to noise exposure. As described by the American College of Occupational and Environmental Medicine (ACOEM) and the predictions of ISO1999 and ANSI S3.44 the evolution of HTLs due to noise exposure in the 0.5, 1 and 2 kHz are less than the HTL evolution in the 3, 4 and 6 kHz. Using the global exposure level, the predictions of both ISO1999 and ANSI S3.44 standards can be performed to determine if the estimated REEL is conform to the prediction of ISO and ANSI standards. This can lead to the identification of non-conforming HTL evolution between certain frequencies to those of ISO and ANSI by comparison with a set of serial audiograms of an individual such as those presented FIG. 6. Such identification of a non-conforming frequency evolution can be verified by analyzing potential causes, such as inadequate locale, decalibration of audiometer, improper use of HPDs, use of drug or alcohol by subject tested, malingering, or by intrinsic or extrinsic factors such as personal pathologies, genetic factors or noisy hobbies. After eliminating technical causes, of interest is the pattern analysis which is in the domain of medical expertise (Step 614). Several patterns described in the literature are analyzed for the identification of HTL evolution patterns not compatible with NIHL such as low-slopping, mid-slopping, high-slopping, strial and flat audiograms. If a series of audiograms for an individual is available, then the entire series of audiograms may be analyzed by the health professional. The non-conforming evolution of HTLs at certain frequencies can lead to the identification of problem cases not related to NIHL, Step 621.

Several factors of intrinsic or extrinsic nature can affect the subjective response of an HTL. As a first step, a procedure based on NIOSH recommendations is used. The major criteria for exclusion of an audiogram in the REEL analysis are no response at one or more frequencies, a negative slope in the low frequencies of 500, 1000, and 2000 Hz, a 15 dB variation at any frequency in either ear between two annual tests, a 50 dB variation between two adjacent frequencies, an intra-aural difference of 25 dB for the 500, 1000, and 2000 Hz frequencies and of 40 dB for the 3000, 4000, and 6000 Hz frequencies. These variations between two audiometric tests usually are not compatible with the evolution of HTLs due to noise exposure (Step 621).

The second consideration is the variability of the HTLs inherent to the audiometric technique. The procedure used establishes the HTL in 5 dB steps. A variation of 5 dB, and up to 10 dB in certain cases, is an acceptable variation inherent due to the audiometric technique. While such a variation is acceptable from a clinical aspect, it may invalidate the results of the estimated REEL in certain cases (as demonstrated above). To correct this potential problem, the tests in Step 612, Step 613 are performed.

In Step 614, if a qualified health professional deems the exposure to noise as not likely to have caused the evolution of HTLs from an audiogram to another for a specific period then the REEL value is rejected (Step 621).

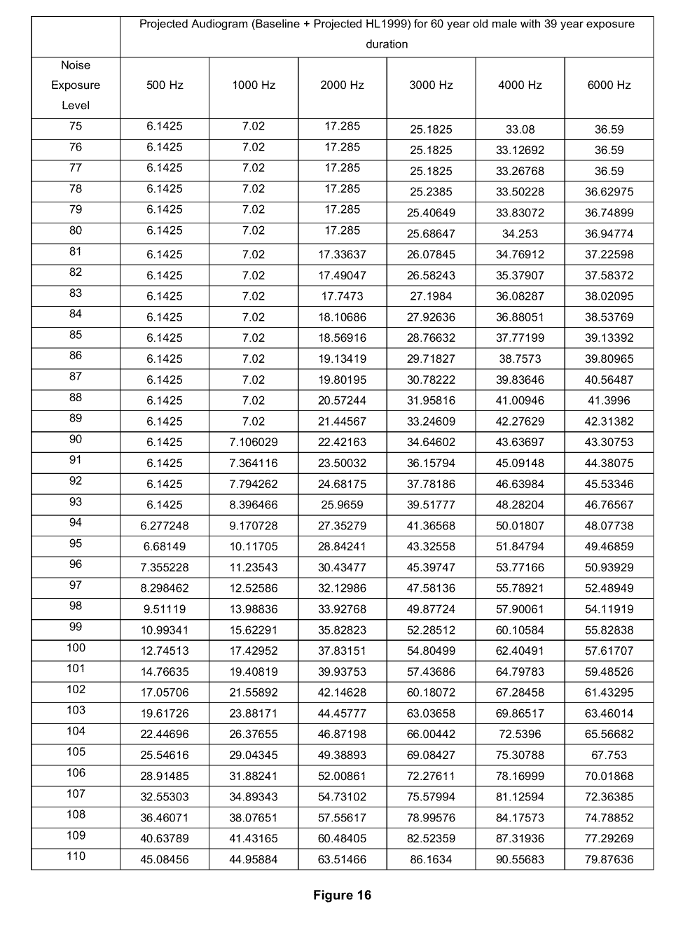

In conjunction with the global REEL value obtained at Step 601, the disclosed method also enables one to calculate the REEL per frequency (Step 602). It is possible to obtain REEL results per frequency by first following the Steps 101 to 501. For Step 501, the fitting involves simply matching the values which would have caused the HTL at a given frequency to vary from Audiogram 1 to Audiogram 2. For example, in evaluating a 60 year old male with 39 years of noise exposure, from FIG. 16, the noise level that would have caused the HTL at 500 Hz to vary from Audiogram 1 to Audiogram 2 is between 103 dB and 104 dB. Similarly, for other frequencies, the results in FIG. 8 are obtained.

The frequency method requires less statistical fitting rigor than the global REEL method since only two data points are being compared to each other. The solution is to pick the noise level that offers the shortest distance between the HTLs of each frequency from a selected projected audiogram and those from Audiogram 2. By comparing the results of the projected audiograms from the ISO1999 or ANSI S3.44 and ISO7029 formula on a frequency basis with Audiogram 2, it is possible to obtain REEL values for each of the projected frequencies (that also need to have corresponding HTLs in Audiogram 2), in this case from 0.5 to 6 kHz.

Using the frequency REEL values obtained in Step 602, the health professional in Step 614 may be able to predict potential intrinsic or extrinsic causes or consider that the evolution of HTLs is caused by an exposure to noise. The first analysis of a health professional would be in relation to the characteristic pattern of NIHL, as stated above, and the second analysis would consist of a pattern analysis in relation to the various patterns related to probabilities of certain intrinsic or extrinsic factors.

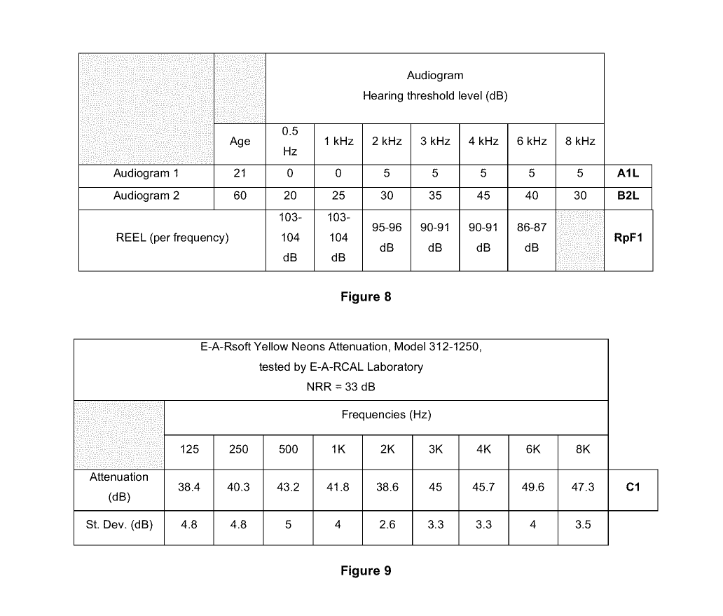

The results from the frequency REEL analysis may be used to compare to the individual frequency attenuation values typically indicated on hearing protection device's data sheets such as those presented in FIG. 9. The results may further be compared to other noise reduction calculation ratios such as the NRR or the HML procedure. The estimated exposure level at each frequency can be obtained in relation to the spectral exposure and laboratory attenuation of each HPD. Using FIG. 8, calculate the difference between the estimated noise exposure and the actual measured noise level at the individual's workstation. According to FIG. 9, there should be a 43.2 dB attenuation at 500 Hz whereas using the REEL shows that there was no actual attenuation perceived by the ear at this frequency. Similarly, whereas the HPD might indicate a reduction of 49.6 dB at 6 kHz, the REEL indicates a reduction of only 17 dB (103 dB−86 dB=17 dB). It is known that the laboratory attenuation does not conform to what is observed in field use of HPDs. It is well documented that field attenuation of HPDs are usually less than 50% of what is observed in laboratory evaluation.

Similarly, it is possible to obtain global attenuation values by subtracting global REEL values such as those presented in FIG. 7 from noise measurement (NEL) values from dosimetry or sound level measurements obtained on the workstation of the individual, if the data is available. For example, if an individual works in an environment measured at 90 dB and his estimated REEL is 83 dB, then it is possible to conclude that the actual in ear attenuation of his hearing protectors was 90 dB−83 dB=7 dB using the NRR approach from the duration between Audiogram 1 and Audiogram 2. Thus, global attenuation values of the HPD used can be obtained.

The suggested method may actually quantify the effectiveness of the HPD used by the individual during the time period between his Audiogram 1 and his Audiogram 2 and warrant a follow up by an industrial hygienist were the difference between the REEL and the known NEL be too little compared to the expected laboratory attenuation of the HPD used.

Active in-ear measurement methods such as the MIRE rely on the assumption that the employee continuously wore his hearing protectors when exposed to noise and he wore them in a proper manner similar to that of the day of his test. The herein proposed method does not make such an assumption since the noise exposure level is estimated based on the evolution of HTLs from audiograms of the individual and is therefore the estimate of the noise exposure level as perceived by the ear. Using REEL, the effectiveness of the hearing protectors used by an individual is evaluated independently of the manner or the time the hearing protector was used. It may be used as a way to validate the noise induced hearing loss risk mitigation policies established by the company. It may on the other hand demonstrate that the protectors are not adequate in reducing the noise exposure or that they are worn incorrectly. The proposed method thus enables to follow up on the effectiveness of the hearing conservation program established at the company and to bring the necessary changes to the workforce in order to reduce the risk of developing noise induced hearing loss.

Once the value for the real ear exposure level (REEL) has been interpreted and accepted for each ear (Steps 611 to 615 and eventually Step 621), the results are prepared and presented in a report (Step 701 to 1003). FIG. 19 shows a sample medical report typically obtained at Step 802, the REEL values allow projections until a selected age such as 65, and STS-OSHA calculations. FIG. 10 is an example only and the information provided in a report may vary.

The information output from using the method may include production of reports (Step 801 to 1003) that may present the REEL estimations for both ears for the individual at 0.5 percentile with a confidence interval with other percentile (Step 801).

Reports such as those in Steps 802 and 803 may be produced that include predicted HTLs at a future date such as the expected retirement age for both ears based on the estimated REEL at selected percentile such as 0.5 and 0.1 assuming noise exposure will remain unchanged for the future years. In such a case, the ISO1999, ANSI S3.44, or ISO7029 projections formulas are used in a conventional way to predict HTLs evolution taking current hearing as the baseline audiogram and projecting HTLs based on continuous exposure to the REEL as the exposure level for a period. The results are prepared and presented in a report in Steps 802, 803, 804, and 1003.

Reports such as those produced in Step 802 and 803 may include clinical comments for individual based on REEL estimations and medical history. They may also include predictions of hearing clinical classifications at the expected retirement age for both ears based on the REEL at 0.5 and other percentiles. They may also include predictions of the averages of the frequencies used for compensation in a selected jurisdiction for both ears based on the REEL at 0.5 and other percentiles.

The REEL prediction may also be used to estimate at which age the evolution of HTLs may render a specific individual ineligible to continue working in a specific workstation in relation to a legislated or specific determined levels required accomplishing such a job. Predictions of HTLs may also predict an employee's non-compliance for certain jobs where specific hearing requirements are required.

Reports such as those in Step 802 may also include STS-OSHA calculations for both ears for the individual. REEL values may also be compared to STS-OSHA to ensure compliance to occupational. Reports may also include predictions of STS OSHA for both ears until the expected age of retirement based on the REEL at 0.5 and other percentiles. They may also include predictions of the ONIHL compensation status based on the REEL at 0.5 and other percentiles.

Reports such as those in Step 803 may also include prediction of the evolution of the average of frequencies used for compensation, such as 0.5, 1K, 2K and 3K (American Medical Association standards) until the expected age of retirement based on serial audiograms of both ears. If monetary compensation data is gathered (Step 903), then information included in reports such as those in Step 803 may be used to produce a financial report (Step 1003) that may include calculations on ONIHL compensation based on the current hearing levels. It may also include predictions of the ONIHL compensations at the expected age of retirement based on the REEL at 0.5 and other percentiles.

Reports such as those in Step 804 may include summary data from reports in Steps 801, 802, 803, and 1003 for all the individuals tested of an entire company workforce. They may also include a summary of retirement predictions for the entire workforce. This will help better assess the risk of hearing loss of the workforce and the degree of hearing loss compensation that could be potentially paid out to the workforce if hearing loss claims are made. This may also be used to justify investment in an up to date hearing conservation program within the company.

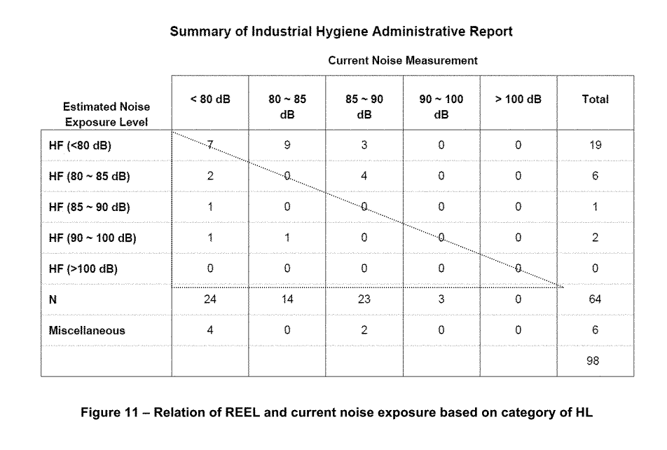

In Step 804, the comparison between the REEL and the NEL at the workstation of an individual may be done on the entire workforce of individuals to obtain a summary report such as the one shown in FIG. 11 in order to identify the number of non-conforming cases. Of note in this figure is the bottom triangular matrix which shows the number of people whose REEL value is higher than the measured noise level at their respective workstation. In FIG. 11, the individuals in the bottom triangular matrix would have to be evaluated by a qualified health professional to determine if the evolution of their HTLs was due to noise exposure or intrinsic/extrinsic factors (Step 614).

Reports such as an administrative report (Step 805) may include calculations of the percentile relationship of the individual according to ISO1999 or ANSI S3.44 based on the REEL and the NEL. They may include comparisons between the REEL value and the NRR or HML attenuation of the HPDs used by the individual to estimate attenuations perceived by both ears (if the employee uses a HPD).

In order to illustrate applications and variations of the above disclosed method, examples are provided hereinafter

### Example 1

In this example, iterations of the projected hearing loss according to ISO1999 performed at Step 401 and Step 501 for noise exposure levels ranging from 75 dB to 110 dB are presented for a male with an exposure duration of 39 years from age 21 (corresponding to audiogram A1L) to age 60 (corresponding to audiogram B2L) according to Formula 2. Detailed calculations are provided for one iteration using a noise exposure of level of 90 dB. Values for □ are taken from the table represented at FIG. 12.

First, the results obtained for the age related hearing loss according to ISO7029 for a 60 year old male are presented in FIG. 13.

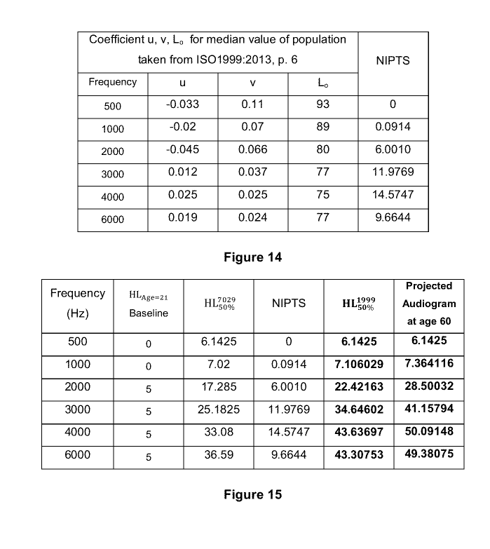

Next, the NIPTS with exposure duration of 39 years is calculated. The results are presented in FIG. 14.

According to the ISO1999, if (L_{ex,8 h}−Lₒ)<0 then NIPTS=0. For such cases, the REEL is 75 dB or less. This corresponds to ISO7029 percentiles of 0.5 or better.

The hearing loss for the six reference frequencies according to ISO1999, ANSI S3.44 and ISO7029 standards for the individual is summarized in FIG. 15.

The projected HTL at a given frequency is the sum of the baseline HTL, here from Audiogram A1L, and the HL1999 predicted value.

Since for the iterations of Step 401, the noise exposure level is to be incremented by approximately 1 dB or less for each iteration, from 75 dB to 110 dB, then, the same procedures as outlined above in this example must be repeated to get 36 projected HL1999, HL7029 results. The results for the projected audiograms are presented in FIG. 16. Note that these results are the sum of the Audiogram 1 HTLs (selected Audiogram A1L) and hearing loss projections according to ISO1999.

According to Step 501, the next step is to use a statistical function to estimate the best fit between Audiogram B2L at the age of 60 and the projected audiograms obtained from the previous step. For this example, the Smooth Huber Loss Function Robust (SHLFR) is used to find the best fit. Using Formula 5, values for X(f) are obtained and are presented in the table shown in FIG. 17.

Finally, using Formula 6, the results presented in FIG. 18 are obtained. From FIG. 18, the smallest Smooth Huber Loss Function Robust (SHLFR) distance between Audiogram 2 and the projected audiogram is 55.36695 and is given by using a noise exposure level of 90 dB. Therefore, based on this example, the REEL obtained at Step 601 for the left ear of this individual is 90 dB. A similar procedure is used to calculate the REEL for the right ear.

Ear exposure to this noise level is most likely to have caused the evolution of his HTLs from his Audiogram 1 at age 21 to his Audiogram 2 at age 60. FIG. 19 represents the superposition of Audiogram 1, Audiogram 2, and the projected audiogram that best fits Audiogram 2, being the one whose corresponding noise level (90 dB) was chosen as the REEL value. However, for this individual, 2 analyses can be performed for 2 different periods. An example of such analysis with serial audiograms is shown in FIG. 7.

### Example 2

In the following example, an example is developed where the exposure duration is greater than 40 years.

If t>40 years, t is set to 40 years according to Formula 4 and the calculations are completed similarly to Example 1 above. For example, assume the same person is of age 62 now, therefore t=41. The results of NIPTS are same as the ones of age 62, but with an exposure duration of only 40 years instead of 41 years (i.e if t>40 years, set t=40 in NIPTS projections and proceed). Note, however that the results for HL7029 are simply dependent on the age of individual for Audiogram 2 and are not affected by the t=40 limitation. The results are found in FIG. 20. It is possible to note from this figure that although the NIPTS factor does not change when the duration is above 40 years, the aging factor predicted from ISO7029 certainly affects the total ISO1999 HL prediction of an individual since it is valid until the age of 70.

If the exposure duration were less than 10 years, one would simply use Formula 3 for the first 10 years and formula 2 for the period of 10 to 40 years.

### Example 3

In the present example, ISO1999 hearing loss projections using percentiles other than the median value are presented.

Using the same example as above, if one uses values of REEL that lie between 10% and 90% of the general population instead of using the median values, the following two different projections per iteration that would be obtained are defined as follows:

$${HL}_{10 \%}^{1999} = {HL}_{50 \%}^{7029} + {NIPTS}_{10 \%} - \frac{{HL}_{50 \%}^{7029} * {NIPTS}_{10 \%}}{120}$$

$${HL}_{90 \%}^{1999} = {HL}_{50 \%}^{7029} + {NIPTS}_{90 \%} - \frac{{HL}_{50 \%}^{7029} * {NIPTS}_{90 \%}}{120}$$

$$\text{Where:}$$

$${NIPTS}_{10 \%} = {NIPTS}_{50 \%} + 1.282 * {d}_{u}$$

$${NIPTS}_{90 \%} = {NIPTS}_{50 \%} + 1.282 * {d}_{l}$$

$$\text{Where:}$$

$${d}_{u} = [ {X}_{u} + {Y}_{u} log ( 39 ) ] {( 90 - {L}_{o} )}^{2}$$

$${d}_{l} = [ {X}_{l} + {Y}_{l} I log ( 39 ) ] {( 90 - {L}_{o} )}^{2}$$

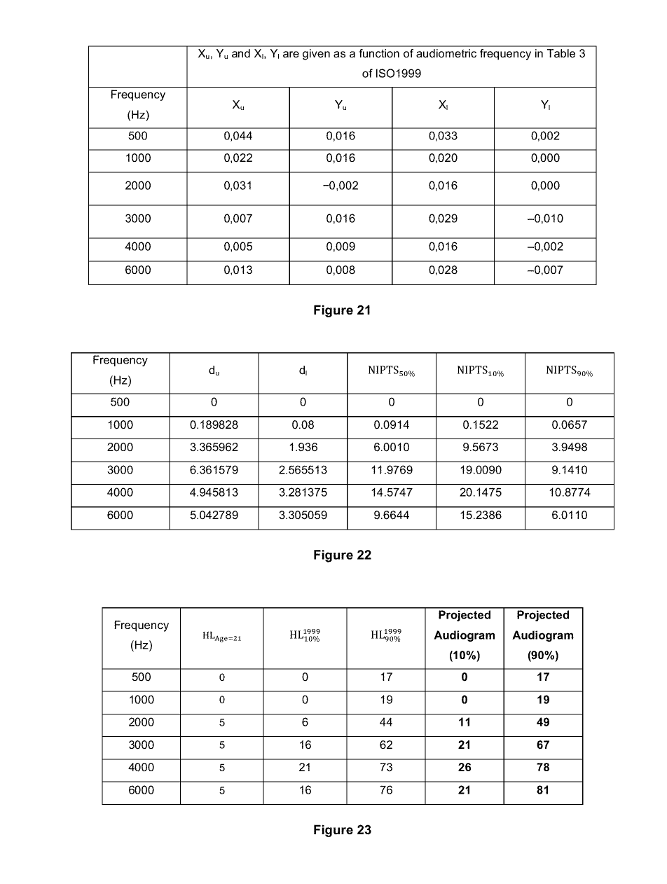

The constants are presented in FIG. 21 and the intermediate results of this example are presented in FIG. 22. The final hearing loss predictions are presented in FIG. 23. Using the SHLFR, we obtain REEL90%=87 dB, and REEL10%=93 dB.

### Variations to the Basic Method and Use of Results

Referring to the selection of two audiograms for Steps 101 to 301 discussed above, it is not necessary to choose the earliest and most recent audiograms of an individual as Audiogram 1 and Audiogram 2 respectively. The disclosed method may be used to estimate the actual noise level perceived by an ear of an individual at any given workstation during a selected period as long as hearing tests are available for that period. If an audiogram taken right before a person started working at a new workstation is available and another one is available at another period, the method may be applied on these two audiograms to determine the REEL during that time period. Although dosimetry and sound level measurements are used to determine the NEL of the work environment, the disclosed method is actually the most accurate in terms of determining the noise exposure as perceived by the ear. This information is useful for risk mitigation of hearing loss in industry such as period A and period B. Using the data from FIG. 1 and the characteristics of the same individual as in Example 1, performing the steps of the herein disclosed method leads to the REEL values shown in FIG. 7. For this individual, from age 21 to age 54 for company A, the calculated REEL is 97 dB and from age 54 to age 60 for company B, the calculated REEL is 75 dB. Whereas, from age 21 to age 60, a REEL of 90 dB was obtained, according to Example 1.

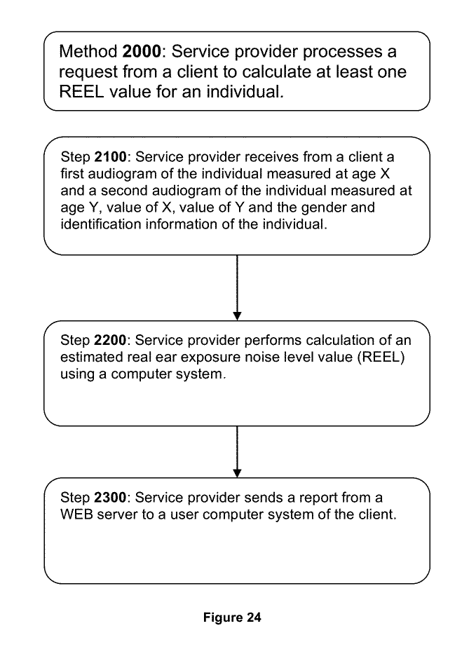

Referring to FIG. 24 according to an embodiment, there is further provided a method for determining a REEL associated as a cause of an evolution of hearing acuity of an individual. According to a first embodiment of the method 2000 illustrated in FIG. 24, a service provider would process a request from a client to calculate the REEL value for an individual the values of the REEL for several employees of a company. Therefore, the following steps are involved:

First, at Step 2100, the service provider receives from a client a first audiogram and coordinates of the individual measured. Such information may be supplied by the client from a user computer station to a WEB server of the service provider, using an Internet platform.

The service provider performs calculation of an estimated real ear exposure noise level value (REEL). The service provider may the method for determining a real ear exposure level as described in the foregoing disclosure. The service provider sends a report from a WEB server to a user computer system of the client, the report comprising information about the individual and the calculated estimated noise exposure level value.

The provider would then provide individual estimated REEL results according to the disclosed method and present the information in reports such as those presented in Steps 801 to 1003 of FIG. 2.

In an embodiment, the client is a company and the service provider may calculate and provide REEL values for several employees of that company. The provider may also present group reports in which a depersonalized global evaluation of the company workforce with estimated REEL results is presented in a report. The provider may also calculate more than one REEL value for a given individual, such as one REEL value per ear and/or REEL values corresponding to more than one pair of audiograms.

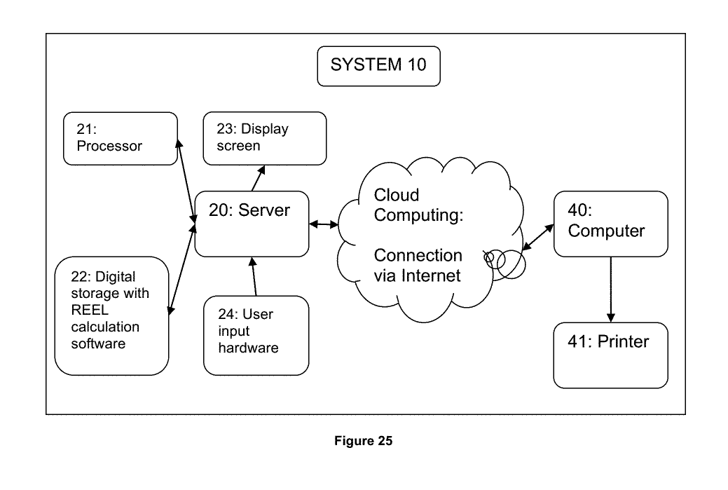

In order to perform that method 2000, a system may be provided according to a further embodiment. An example of such a system is illustrated at FIG. 25. The system 10 comprises a server 20 of a service provider, the server being connected to the Internet and thereby remotely accessible and comprising a data processing unit 21 in digital communication with a digital storage unit 22 storing a computer program enabling calculation of a REEL value from data comprising a first audiogram Such user interfaces may alternatively be remotely located in a networked provider computer workstation. The system 10 may further comprise a client side computer system 40 also connectable to the Internet to access the service provider server 20. The client computer system 40 may further comprise a printing unit 41 to enable printing of a REEL report from data receivable from the service provider server 20.

Therefore, according to the method 2000 and the system 10 provided herein, a client could enter audiometric data, such as audiogram HTLs, in a service website on an internet browser via an internet connection and the provider would complete REEL calculations as proposed in the disclosed method based on the input information sent by the client. The calculations would be performed in the provider's server via cloud computing or using a computing method to the same effect and the results would then be communicated back to the client in the form of a data report.

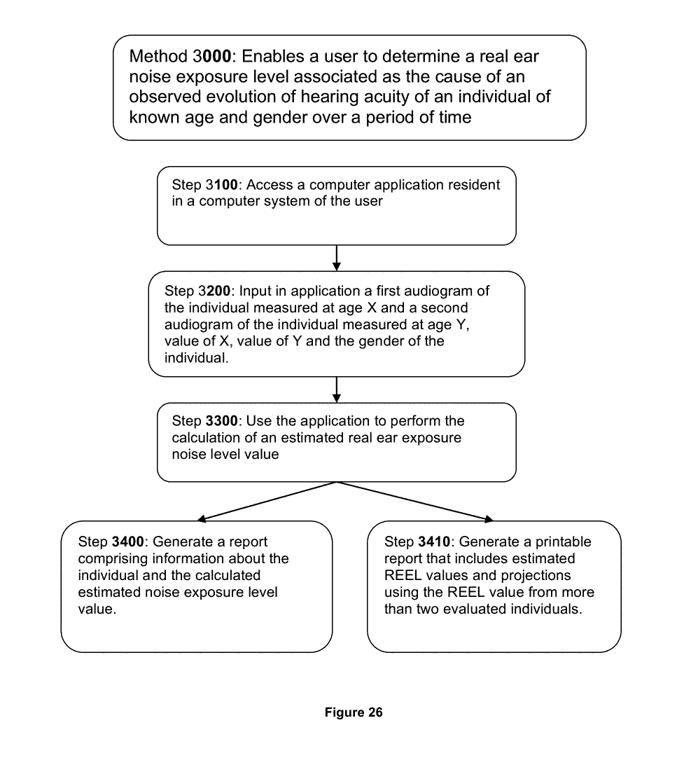

According to an alternate embodiment, a method is provided, as illustrated in FIG. 26, whereby the client would obtain computer application software capable of calculating REEL values according to the disclosed method and use it to obtain estimated REEL values without requiring direct access to a service provider server according to the method 2000 described above. Accordingly, referring to FIG. 26, a method 3000 is generally provided to enable a user to determine a real ear noise exposure level associated as the cause of an observed evolution of hearing acuity of an individual of known gender over a period, the method comprising: Step 3100: Accessing a computer application resident in a computer system of the user or under the user's control; Step 3200: inputting in the application a first audiogram of the individual measured at age X and a second audiogram of the individual measured at age Y, value of X, value of Y and the gender of the individual. Step 3300: use the application to perform the calculation of an estimated real ear exposure noise level value. The method may further comprise a Step 3400: generating a report comprising information about the individual and the calculated estimated noise exposure level value.

Accordingly, the method 3000 enables a user to input audiometric data by providing a first set of audiometric hearing threshold levels at 0.5, 1, 2, 3, 4, 6 kHz of an individual measured at age X and a second set of audiometric hearing threshold levels at 0.5, 1, 2, 3, 4, 6 kHz of the individual measured at age Y. The user would then input the gender, age X, age Y, or directly a time period equal to Y−X to be used in the ISO11999 or ANSI S3.44 HTL projection formulas calculated by the computer program application. An estimated REEL value could then be generated according to the disclosed method and output to the user on a screen or a device to this effect. The estimated REEL value for an individual is presented in a report that would include information about the individual and the estimated REEL values associated with the evolution of the HTLs observed in a pair of his audiograms as inputted. Estimated REEL values may be presented for each available pair of audiograms for an individual that has more than two available audiograms, such as a series of audiograms. In the presence of a series of audiograms, the report may be generated, including an analysis by qualified persons such as a health professional comparing the evolution of the individual's HTLs and the estimated REEL values.

According to a further embodiment, the method 3000 may further include a Step 3410 wherein a report may be printed that includes estimated REEL values and projections using the REEL value from more than one evaluated individual. The report may also include averages of REEL values of similar workstations and various statistical measures applied to the evaluated individual(s) from a client or clients.

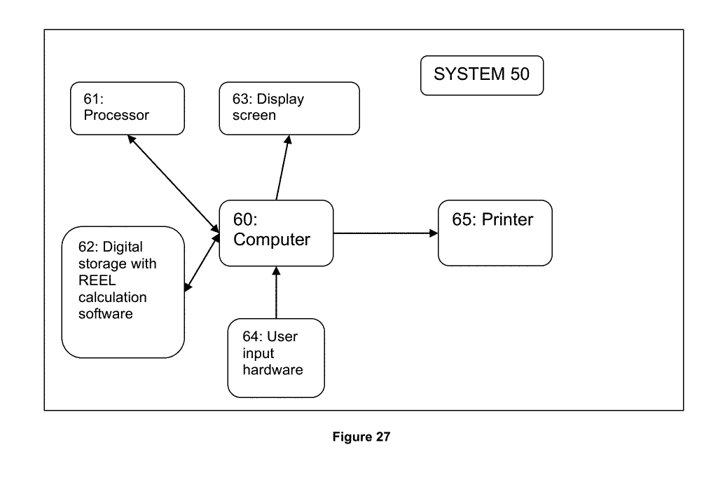

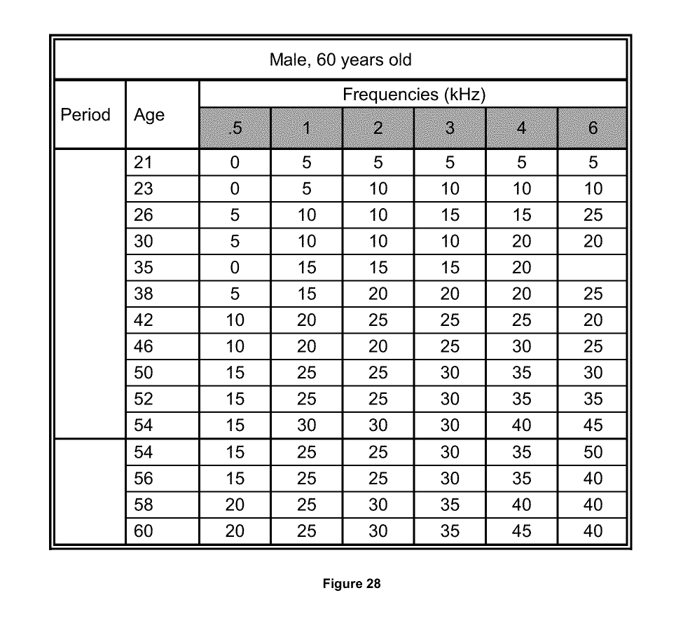

In order to perform method 3000, a system may be provided according to a further embodiment. An example of such a system is illustrated at FIG. 27. The system 50 comprises a computer system 60 (local or remotely accessible) controlled by a user, the computer system comprising a data processing unit 61 in digital communication with a digital storage unit (internal or external memory) 62 storing a computer program enabling calculation of a REEL value from data comprising a first audiogram of the individual measured at age X and a second audiogram of the individual measured at age Y, value of X, value of Y and the gender of the individual. The user computer system 60 may further comprise user interfaces such as a display screen 63 and input hardware such as a keyboard 64. Such user interfaces may alternatively be remotely located in a networked computer workstation. The system 50 may further comprise a printing unit 65 to enable printing of a REEL report from data generated by the computer program stored in the storage unit 62.

In conclusion, the present disclosure provides methods and systems for elucidating the cause of hearing loss in individuals, which obviate at least some of the limitations and drawbacks of the prior art methods and systems.

While preferred embodiments have been described above and illustrated in the accompanying drawings, it will be evident to those skilled in the art that modifications may be made thereto without departing from this disclosure. Such modifications are considered as possible variants comprised in the scope of the disclosure, as described by the following claims.

1. A method for estimating a noise exposure level to which an individual having an age and a gender has been exposed during an exposure duration, an exposure to the noise exposure level having induced an evolution of hearing threshold value (HTL) of the individual, the method comprising:
obtaining a test audiogram of the individual measured at a time of testing;
obtaining a reference audiogram of the individual at a beginning of the exposure duration;
inputting the individual's gender, the individual's age at the time of testing, and the exposure duration at the time of testing in a prediction formula;
calculating, using the prediction formula and the reference audiogram, a plurality of projected audiograms each associated with a possible noise exposure level;
for each of the plurality of projected audiograms, comparing the projected audiograms with the test audiogram;
performing a curving fitting operation to select one audiogram, among the plurality of projected audiograms, that best fits the test audiogram; and
selecting the noise exposure level associated with the selected projected audiogram as an estimated noise exposure level to which the individual was exposed having induced the evolution of hearing threshold value (HTL) of the individual.

2. The method of claim 1, wherein obtaining a reference audiogram comprises producing a baseline audiogram and assigning the baseline audiogram to the reference audiogram, wherein the baseline audiogram corresponds to an audiogram of an otologically normal individual before exposure to the noise exposure level.

3. The method of claim 1, wherein the obtaining a reference audiogram comprises obtaining an initial audiogram of the individual by measuring at the beginning of the exposure duration to the noise exposure level and assigning the initial audiogram to the reference audiogram.

4. The method of claim 3, wherein the initial audiogram is based on tests at a plurality of frequencies.

5. The method of claim 4, further comprising:
producing a third audiogram of the individual by setting the HTL at each of the plurality of frequencies to a value specified in an aging factor table for a 0.5 percentile for each of the frequencies, for an individual of the individual's gender and the individual's age, thereby providing a third audiogram corresponding to a normal hearing acuity at a beginning of the exposure duration of an individual of the individual's gender and individual's age having a hearing loss solely affected by aging;
comparing the HTL of the selected projected audiogram with those of the third audiogram; and
if two or more HTLs of the selected projected audiogram are higher than the HTLs of the third audiogram on a frequency by frequency basis for two or more frequencies, then it is established that the initial audiogram is invalid and a valid noise exposure level could not be obtained from using the initial audiogram.

6. The method of claim 1, wherein the initial audiogram provides a specific HTL for each of a plurality of frequencies, the method further comprising:
comparing each specific HTL with values provided in an aging factor table for the individual's gender and at the individual's age at the time of testing for percentile values ranging from 0.1 to 0.9 on a frequency by frequency basis; and
if two or more HTLs of the initial audiogram correspond to percentile values higher than 0.5 for the same frequencies, then considering the initial audiogram to be invalid and that a valid noise exposure level could not be obtained from using the initial audiogram.

7. The method of claim 1, wherein the reference audiogram is tested by setting an HTL at each of a plurality of frequencies to a value specified in an aging factor table for the 0.5 percentile for each of the plurality of frequencies.

8. The method of claim 7, further comprising obtaining the aging factor table from standard ISO7029.

9. The method of claim 1, wherein setting a first noise exposure level to the estimated noise exposure level, wherein a second noise exposure level is selected based on a second audiogram tested by setting an HTL at each of a plurality of frequencies to a value specified in an aging factor table for the 0.5 percentile for each of the plurality of frequencies, and
the method further comprising comparing the first noise exposure level to the second noise exposure level and selecting the higher of the first noise exposure level and the second noise exposure level as the estimated noise exposure level.

10. The method of claim 1, further comprising obtaining the prediction formula from the ISO1999 or ANSI S3.44 standard.

11. The method of claim 1, wherein the selected noise exposure level is selected from the group comprising level values ranging from 75 to 110 dB.

12. The method of claim 1, wherein selecting the projected audiogram comprises using a statistical data fitting formula.

13. The method of claim 12, wherein the statistical data fitting formula comprises one of: a Smooth Huber Loss Function formula; a robust class of formulas; and a Smooth Huber Loss Function Robust formula.

14. A system for estimating a noise exposure level to which an individual having an age and a gender has been exposed during an exposure duration, an exposure to the noise exposure level having induced an evolution of hearing threshold value (HTL) of the individual, the system comprising:
a user interface for inputting a test audiogram of the individual measured at a time of testing, the individual's gender, the individual's age at the time of testing, the exposure duration at the time of testing, and identification of the individual;
a computer digital storage storing a computer program for performing calculation of an estimated noise exposure level deemed of having induced an evolution of hearing threshold value (HTL) of the individual over the exposure duration, wherein performing the calculation comprises the steps of:
calculating, using a prediction formula and a reference audiogram of the individual at a beginning of the exposure duration, a plurality of projected audiograms each associated with a possible noise exposure level;
for each of the plurality of projected audiograms, comparing the projected audiograms with the test audiogram;
performing a curving fitting operation to select one audiogram, among the plurality of projected audiograms, that best fits the test audiogram; and
selecting a noise exposure level associated with the selected projected audiogram as the estimated noise exposure level to which the individual was exposed having induced an evolution of hearing threshold value (HTL) of the individual;
a processing unit for executing the computer program; and
an output device for outputting information about the individual and the estimated noise exposure level.

15. The system of claim 14, wherein the computer program is further for producing a baseline audiogram, wherein the baseline audiogram corresponds to an audiogram of an otologically normal individual before exposure to the noise exposure level.

16. The system of claim 14, wherein the user interface is further for inputting an initial audiogram of the individual measured at a time before the exposure to the noise exposure level.

17. The system of claim 14, further comprising:
a service provider server for receiving data inputted at the user interface, and
service provider computing facilities comprising the computer digital storage, the processing unit and the output device, wherein the service provider computing facilities further comprise communication means for transmitting the estimated noise exposure level to the user interface along with the information about the individual.

18. A computer method for estimating a noise exposure level to which an individual having an age and a gender has been exposed during an exposure duration, an exposure to the noise exposure level having induced an evolution of hearing threshold value (HTL) of the individual, the method comprising:
providing to a user access to a computer application resident in a computer;
receiving from the user a test audiogram of the individual measured at a time of testing, the age of the individual at the time of testing, the exposure duration at the time of testing, and the gender of the individual;
using the computer application to perform the steps of:
calculating, using a prediction formula and a reference audiogram of the individual at a beginning of the exposure duration, a plurality of projected audiograms each associated with a possible noise exposure level;
for each of the plurality of projected audiograms, comparing the projected audiograms with the test audiogram;
performing a curving fitting operation to select one audiogram, among the plurality of projected audiograms, that best fits the test audiogram; and
selecting a noise exposure level associated with the selected projected audiogram as the estimated noise exposure level to which the individual was exposed having induced the evolution of hearing threshold value (HTL) of the individual; and
generating a report comprising information about the individual and the estimated noise exposure level.
